(19) United States
(12) Patent Application Publication
(10) Pub. No.: US 2011/0015513 A1
(43) Pub. Date: Jan. 20, 2011

(54) TRANSPORTABLE DIGITAL AUTONOMOUS UNIT TO PERFORM ANTHROPOMETRIC MEASUREMENTS, OF TACTILE MANUAL LOCALIZATION, GEOMETRICALLY BASED ON THE PROJECTION OF POINTS IN A CARTESIAN (VIRTUAL) PLANE, INCORPORATING TECHNOLOGY OF SENSORS AND SOFTWARE

(76) Inventor: Miguel Angel Murá Yanez, Santiago (CL)

Correspondence Address: STRATTON BALLEW, 213 S 12TH AVE, YAKIMA, WA, 98902, US

(21) Appl. No.: 12/083,897

(22) PCT Filed: Oct. 18, 2006
(86) PCT No.: PCT/IB2006/004235

§ 371 Date: Sep. 30, 2010

(30) Foreign Application Priority Data
Oct. 18, 2005 (CL) 2734 2005

(51) Int. Cl. A61B 5/107; A61B 6/00

(52) U.S. Cl. 600/407; 600/587

(57) ABSTRACT

A systematic procedure and device able to measure the body dimensions of persons, both young and adult, relating to anthropometry, and more concrete methods for anthropometric studies. The device includes an autonomous, transportable digital system to carry out the anthropometric measurements, which permits and facilitates the location and collection of the anthropometric points that are extracted of the individual (8) to be measured. Specifically, the device includes a posture correction element (3), a Cartesian framework (4), data transmitters (4.3 and 4.4), operating software (5), and an information processing component that is preferably a notebook type of computer (5.1).

### DESCRIPTIVE REPORT

The present invention refers to a systemic procedure and a device capable of measuring the corporal dimensions of people (children and adults).

The application field of the invention belongs, therefore, to the anthropometry, and in a more concrete way to the anthropometric studies, where the measuring equipment is that that performs the measurement, and who are measured, generally are populational segments or profiles, as for example: children of determined age rank or workers of a determined company, etc. The representative individuals are measured, with certain purposes and criteria, characteristic of the study. Studies formed both by an individual and a great volume of these. It constitutes an instrument capable of, and destined to, extract a data base, which synthesizes and represents in quantitative terms. Data that corresponds to an average, percentile, etc.

### BACKGROUND OF THE INVENTION

Taking into consideration that the population in dimensional terms is heterogeneous in the globe, and is in constant evolution, and its direct implication, insofar as this one is coupled, manipulated and is contained in the objects, is why the measuring and knowing the corporal measurements is indispensable for who involve the dimensional variable in their occupation.

This is the direct situation of the product designers, architects, designers of garments and clothes, prosthesis design, evaluation and studies of high performance in the sports field, institutions concerned about the working conditions, etc. Besides, today is where the technologic transference in the globalization frame, where this data becomes more and more necessary and valued, both locally and internationally.

There are 31 or 37 anthropometric measurements (Based on: Précis de physologie du travail, Notions d'ergonomie, J. Scherrer et collaborateurs, 2^{nd} Edition, Masson Editorial, Paris, 1967, 1981 and Victoria Ratinoff Ferrera, Professor of Ergonomics and Chief of the Ergonomics Laboratory of the Metropolitan Technological University, Regular Member of the National Ergonomic Commission), based on anthropometric points with reference to the skeletal structure (external and insides) and positions of anthropometric attention for the individuals who are measured (standing up, seated and arm span).

In general, the poblational segment or profile should be measured at the place where they perform their duties, for example, the scholars at school, the worker at the company, etc., being necessary to transport both the available measuring equipment and the equipping.

Among the digital methods, there are different appliances capable of recognizing the body and its structures with a high level of precision. For the case of the scanner, they are not transportable, and besides the volume of information for this kind of studies, although it is not necessary, they posses a scale and subsequent work that is not appropriate. The photometric cameras, although seem the best solution because of their instantaneity and today technologically more precise, they invade the measured user, in terms of exposing his/her corporal privacy to a register and its subsequent processing.

The manual method measurements, where the detection and localization of the anthropometric point is tactilely (manually) performed, being more reliable and exact, but require more work, time and a staff of several people.

The set of instruments utilized with the manual method, is mainly formed by: Martin type and reticulate Anthropometer, extensible calibrators, sliding compass, and flexometers (hoists).

It presents problems such as:
Operating fatigue because of instability of the instrument.
Deficient manipulation in regard to stability of the instrument and reliability of reading.
Slow performance of measurement, because it is necessary to coordinate 3 people (measurer, annotator or helper and the measured person).
Substantive aggressiveness (acute edges, temperature, etc.) of the set of instruments, taking into consideration that, in general, the individuals are measured almost without clothes or garments.
Sources of error, both the transmission of data by oral means and the manual transcription.

Logistically, there are physical-administrative problems, related with the disposition of the physical place by the institution from which the measured individuals are extracted (simples) due to the uncertainty of the time required to perform the measurements.

The areas furnished for the performance of the dimensional record, generally are not the most appropriate ones, insofar as, they present irregularities on floor level, walls and structure in general, and certainly, the lighting, increasing sources of error that add accumulatively and progressively, to the essential of the measurement, the data base and the time needed in order to perform it.

Therefore, regarding the technique, there is a need of a device that can obtain efficient anthropometric data on site in a systemic way, of easy utilization.

### PURPOSES OF THE INVENTION

The purpose of this Project consists in obtaining qualitative data of the anthropometric measurements on site, responding to the reliability, celerity and space availability requirements. Systematically taking into consideration: transport, deployment and leveling of the product, orientation and postural adjustment (measured person), tactile manual localization (measurer person), record and digitalization, storage; processing, post-processing and diffusion of the anthropometric data.

Eventually, this may allow boosting and creating data bases, facilitating the corporal dimensional parameters of the user group, in a reliable and precise way, within a projectional process (in the case of the designer). At the same time, this will contribute the designer and other disciplines (medicine, sports, clothes) to qualitatively evaluate the human dimensional factor.

### BRIEF DESCRIPTION OF THE DRAWINGS

Other characteristics and benefits of the invention will be made evident as of the description that follows its preferred performance, given only as an illustrative example and not limited, with reference to the attached drawings, in which:

FIG. 1 shows the diagram of the procedure constitutive of the present invention;

FIG. 2 illustrates a lateral view of the invention's protectors that are being transported in a capacity vehicle;

FIG. 3 illustrates in lateral view the invention's Protectors manually transported;

FIG. 4 shows a view in front perspective of the invention's Protector, containing Postural Rectifier;

FIG. 5 shows a view in rear perspective of the invention's Protector, containing the Postural Rectifier;

FIG. 6 shows a view in front perspective of the invention's folded Postural Rectifier;

FIG. 7.1 shows a view in rear perspective of the invention's Postural Rectifier unfolding itself;

FIG. 7.2 shows a view in front perspective of the invention's unfolded Postural Rectifier;

FIG. 7.3 shows a view in rear perspective of the invention's unfolded Postural Rectifier;

FIG. 8 shows a view in front perspective of the invention's Cartesian Protector, in closed stage, containing the Cartesian Frame;

FIG. 9 shows a view in rear perspective of the invention's Cartesian Protector, in closed stage, containing the Cartesian Frame;

FIG. 10 shows a view in front perspective of the invention's Cartesian Protector, in open stage, in which the folded Cartesian frame is extracted;

FIG. 11 shows a view in front perspective of the invention's Cartesian Frame, in folded stage;

FIG. 12 shows a view in rear perspective of the invention's Cartesian Frame, in folded stage;

FIG. 13.1 shows a view in front perspective of the invention's Cartesian Frame, being unfolded;

FIG. 13.2 shows a view in front perspective of the invention's Cartesian Frame, in unfolded stage;

FIG. 14 shows a view in front perspective of the invention's Postural Rectifier and the Cartesian Frame, in coupling phase;

FIG. 14.1 shows a detailed view in front perspective of the invention's Upper Support;

FIG. 14.2 shows a detailed view in front perspective of the invention's Receptor;

FIG. 15 shows a view in front perspective of the Postural Rectifier, Cartesian Frame and a Notebook of determined characteristics, in data transference and leveling phase, for the eventual utilization of the invention;

FIG. 15.1 shows a view in front perspective of the invention's Postural Rectifier, Cartesian Frame and a Notebook of determined characteristics, in measurement transference phase utilized by both users;

FIG. 16 shows an illustrative detail of the invention's view in front perspective, corresponding to the procedure of standard measurement performed to the measured user (8) by a professional who studies anthropometry, measured user (9);

FIG. 17 shows a view in frontal perspective, in which a standard measurement is performed, where the invention's Cartesian Frame is rotated in Frontal View Position;

FIG. 18 shows a view in frontal perspective, in which a standard measurement is performed, where the invention's Cartesian Frame is rotated in Right Lateral View Position;

FIG. 19 shows a view in frontal perspective, in which a standard measurement is performed, where the invention's Cartesian Frame is rotated in Rear View Position;

FIG. 20 shows a view in frontal perspective, in which a standard measurement is performed, where the invention's Cartesian Frame is rotated in Left Lateral View Position;

FIG. 21 shows an invention's view in upper cut, in which a Horizontal Oblique Measurement is performed;

FIG. 22 shows a frontal orthogonal view, where the invention's Cartesian Frame is rotated in sagittal position, in which a Vertical Oblique Measurement is performed;

FIG. 23 shows an invention's lateral orthogonal view, where the Cartesian Frame is rotated in sagittal position, in which an a Horizontal Aligned Measurement is performed;

FIG. 24 shows an invention's view in lateral perspective, in which a standard measurement is performed, arranged in order to receive standing up posture;

FIG. 25 shows an invention's view in lateral perspective, in which a standard measurement is performed, arranged in order to receive the sedent (in sitting position) posture;

FIG. 26 shows an invention's view in lateral perspective, in which a standard measurement is performed, arranged in order to receive the arm span position;

FIG. 27 shows a view in front perspective of the quartering of the invention's main components;

FIG. 28 shows an invention's view in upper front perspective of the upper support assembly (3.1);

FIG. 29 shows an invention's view in lower front perspective of the upper support assembly (3.1);

FIG. 30 shows a detail of the invention's upper view A cut of the upper support assembly (3.1);

FIG. 32 shows a detail of the invention's FIG. 31 broken view in frontal view of the upper support assembly;

FIG. 33 shows an invention's view in front perspective of the Column assembly (3.3);

FIG. 34 shows a view in rear perspective of the invention's Column assembly (3.3);

FIG. 35 shows a view in front perspective of the invention's Armrest assembly (3.4);

FIG. 36 shows a view in lower perspective of the invention's Armrest assembly 3.4);

FIG. 37 shows a front perspective of the invention's Seat assembly (3.5);

FIG. 38 shows a view in lower perspective of the invention's Seat assembly (3.5);

FIG. 39 shows a view in front perspective of the invention's Lower Support assembly (3.2);

FIG. 40 shows a view in lower perspective of the invention's Lower Support assembly (3.2);

FIG. 41 shows a view in front perspective of the invention's Frame assembly (4.1);

FIG. 42 shows a view in rear perspective of the invention's Frame assembly (4.1);

FIG. 43 shows a view in front perspective of the invention's Horizontal Axis assembly (4.2;

FIG. 44 shows a view in rear perspective of the invention's Horizontal Axis (4.2);

FIG. 45 shows a view in front perspective of the invention's Master Cartesian Manipulator (4.3);

FIG. 46 shows a view in front perspective of the invention's Slave Cartesian Manipulator (4.4);

### DESCRIPTION OF THE PREFERRED PERFORMANCE

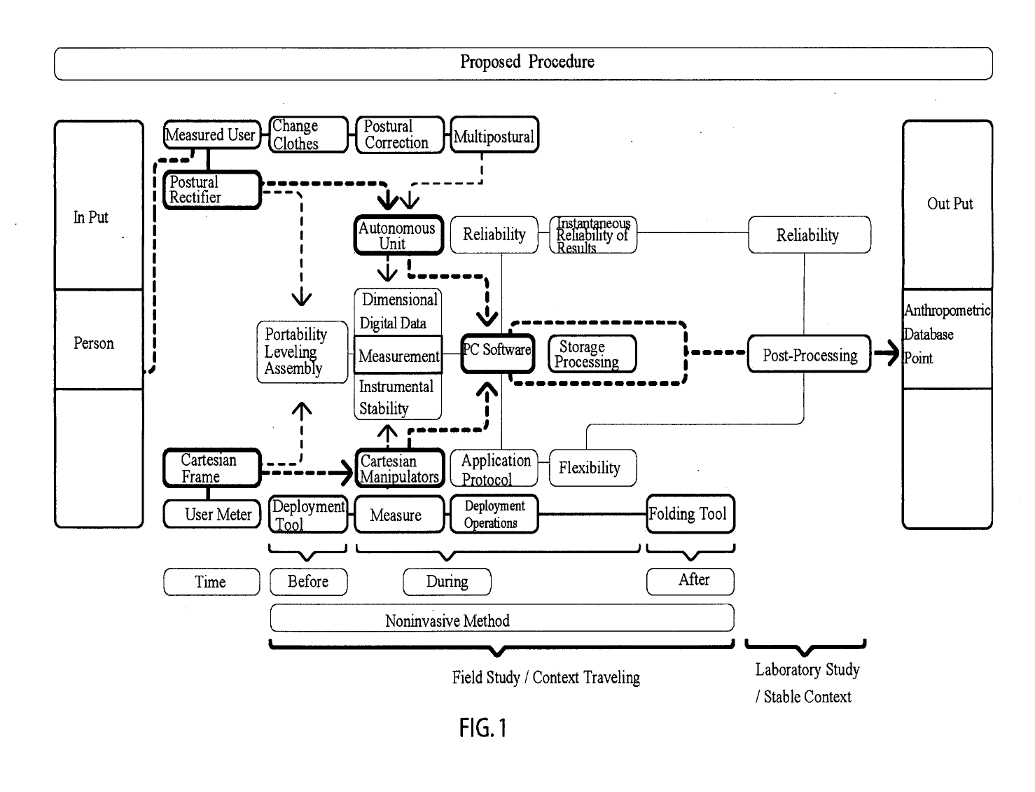

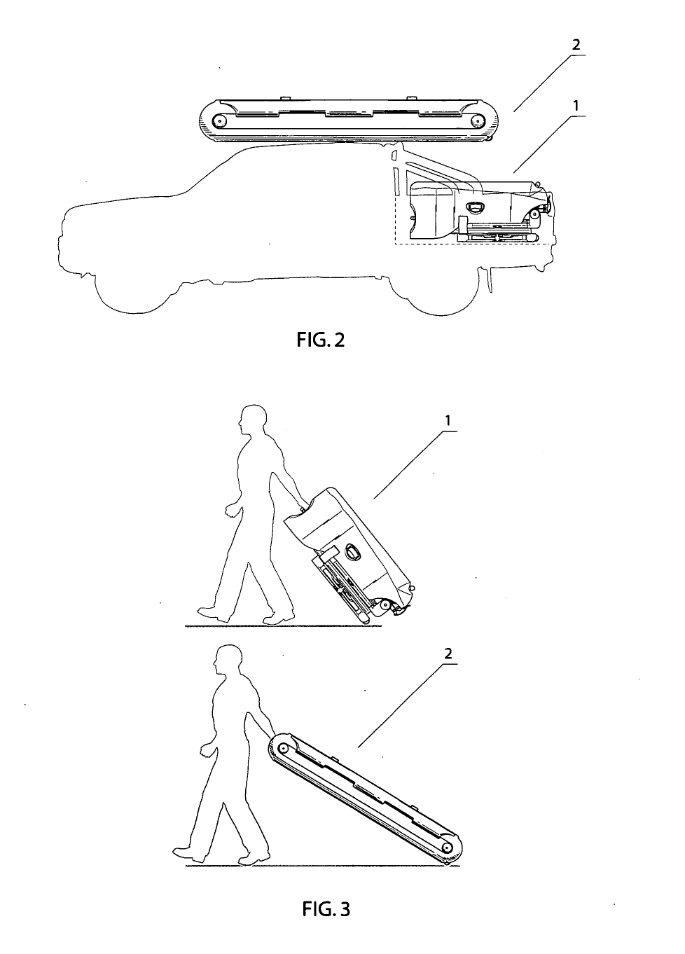

In order to carry out the detailed description of the preferred performance invention's device, continued reference to the drawings' Figures will be made, of which FIG. 1 is the diagram synthesis of the proposed procedure, in whose system we have the individual to measure as input and output of the data base derived from the remarkable anthropometric points that are extracted from this one. It will be constituted by 2 subsystems, the first one, called Postural Rectifier, since in general terms it must orient and receive a postural protocol; and the second subsystem called Cartesian Frame, the one that receives the operational functions of the individual that performs the measurement.

These complementary units are transported in a vehicle of medium capacity, contained by protectors, to be, once on site independently unfolded and to couple afterwards. During the measurement the information is sent to Software, which in digital terms, will allow the record, storage, processing, delivery of results, and post processing.

FIG. 2. In such Figure it is possible to observe, the way in which the Cartesian Frame is arranged in its respective protector (2) on the medium load vehicle, and, in turn, the Postural Rectifier in its respective protector (1).

FIG. 3. In such Figure it is possible to observe the way in which the Postural Rectifier in its respective protector (1) and the Cartesian Frame (2), are respectively moved in their protectors.

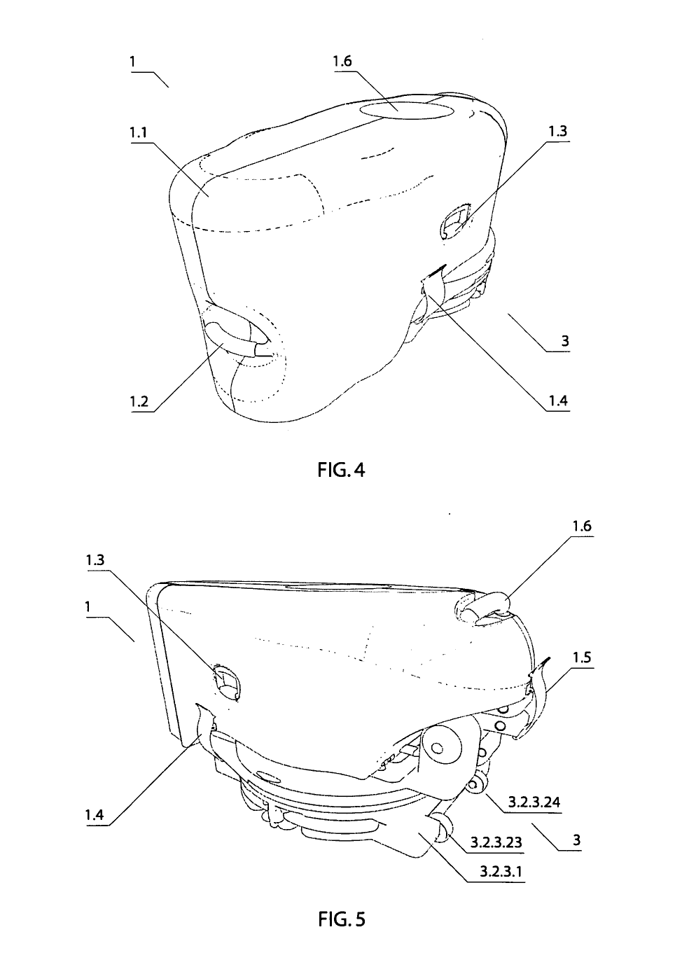

FIG. 4. In such Figure the Postural Rectifier is observed in its respective protector (1), where the greater body or casing (1.1), is able to cover the most susceptible portion from blows of moving and transport, from whose frontal part a concave space is projected where an element of manual pull is horizontally spliced (1.2) for its moving. At both lateral sides a concavity is again projected in which a pivoting cohering element is connected (1.3). In addition, 3 pivot hole-anchorages are incorporated into the protector, (1.4) which are able to secure the union between this one and the Postural Rectifier, one of which is appreciated in all its expression in the following Figure. Finally, a circular cut is projected in the upper zone (1.6) for the incorporation of graphics.

FIG. 5. In such Figure the third pivot hole-anchorage belonging to the casing (1.5) is observed. The rear concavity in which a cohering element is horizontally lodged (1.6). And, finally belonging to the Postural Rectifier, in a portion removed from the base (3.2.3.1) two cylinders (3.2.3.23) and (3.2.3.24) projected towards its interior and individually connected.

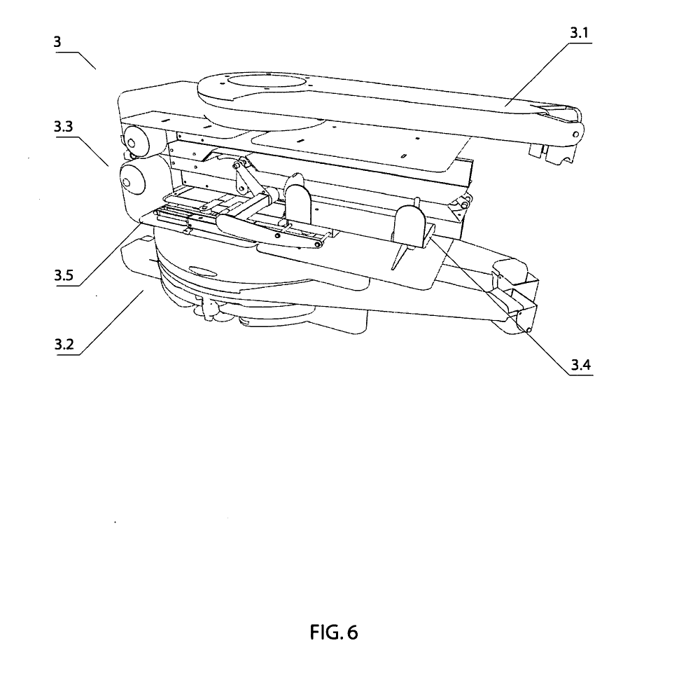

FIG. 6. In such Figure the Postural Rectifier without its protector totally folded, is observed where; the Upper Support (3.1), the Lower Support (3.2), the Column (3.3), the Armrest (3.4) and the Seat (3.5), are folded, backed and retracted.

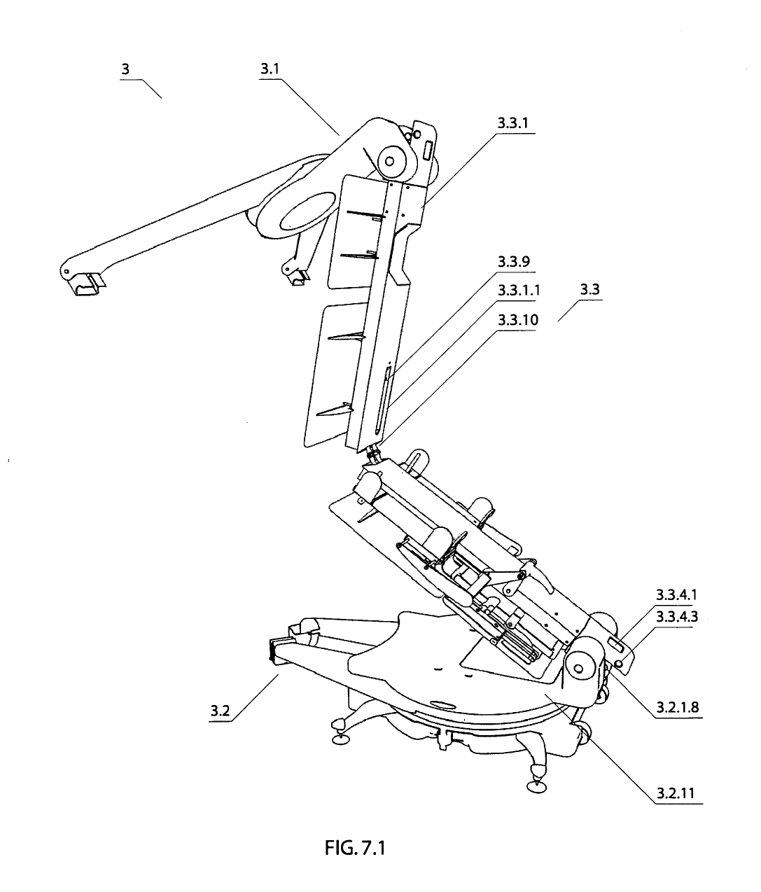

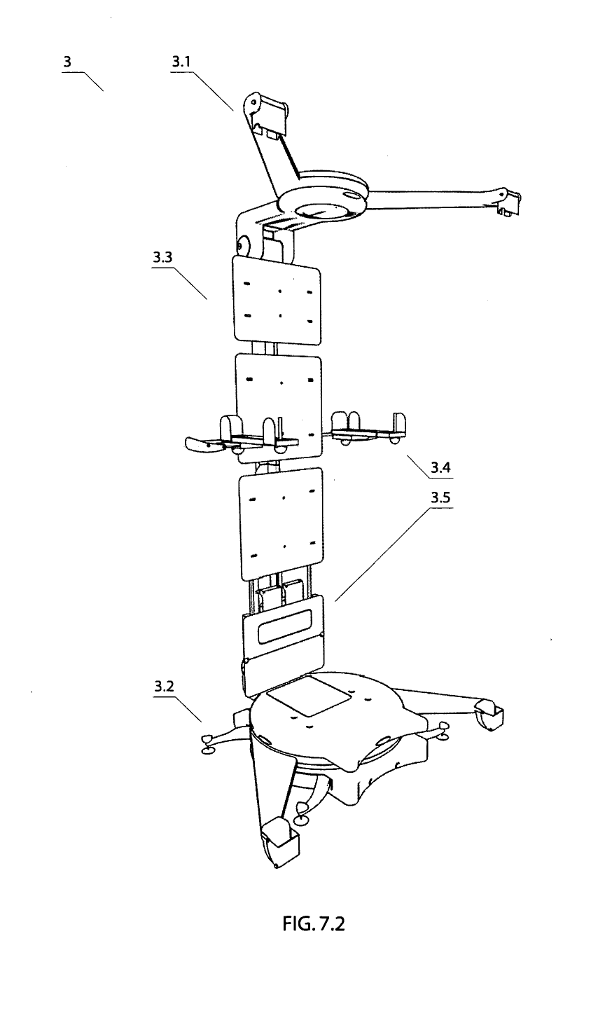

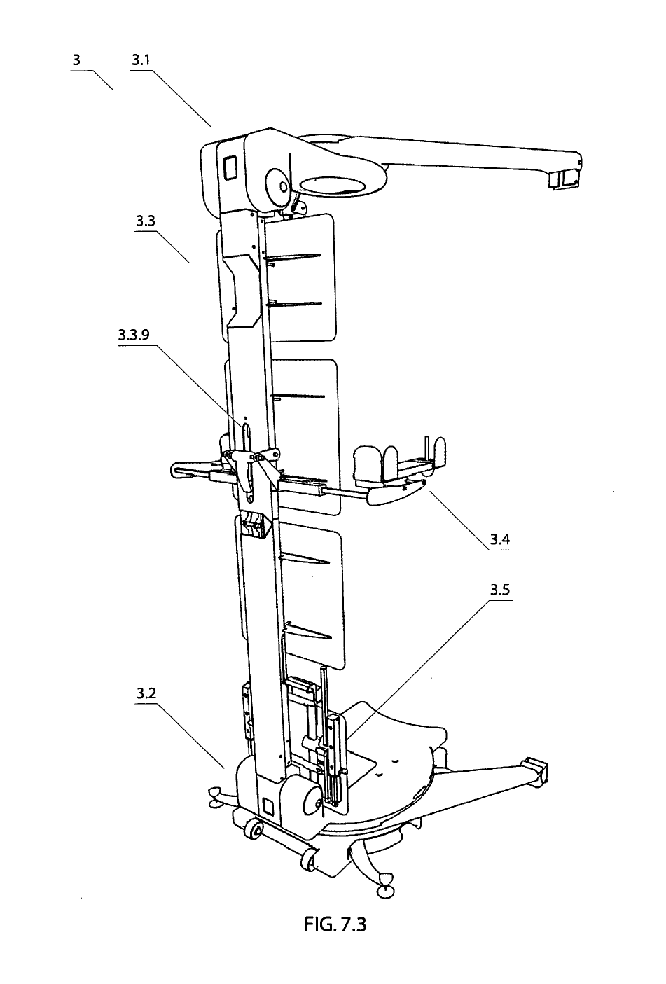

FIG. 7.1. In such Figure, it is possible to observe a smaller body (3.3.9) able to slide in the extended portion (3.3.1.1) removed from the drained greater body (3.3.1). Hinging (3.3.10), smaller body connector of the 2 greater drained bodies (3.3.1) and (3.3.2), these greater bodies are able to pivot in horizontal and perpendicular axis to their guidelines of the spreading out movement. Ends and terminals to these greater drained bodies lodge an activator (3.3.4.1), an effector (3.3.4.3) that at its moment of adjustment is introduced in counterform (3.2.1.8), the latter lodged in platform (3.2.11)

FIG. 7.2. In such Figure the Postural Rectifier in front perspective view totally unfolded is observed, where; the upper support (3.1) and the lower support (3.2) are united and articulated by the column (3.3), in which the armrest (3.4) and the seat lodge (3.5).

FIG. 7.3. In such Figure the Postural Rectifier in rear perspective view totally unfolded is observed, where; just as in FIG. 7.2; the upper support (3.1) and the lower support (3.2) are united and articulated by the column (3.3), in which the armrest (3.4) and the seat lodge (3.5).

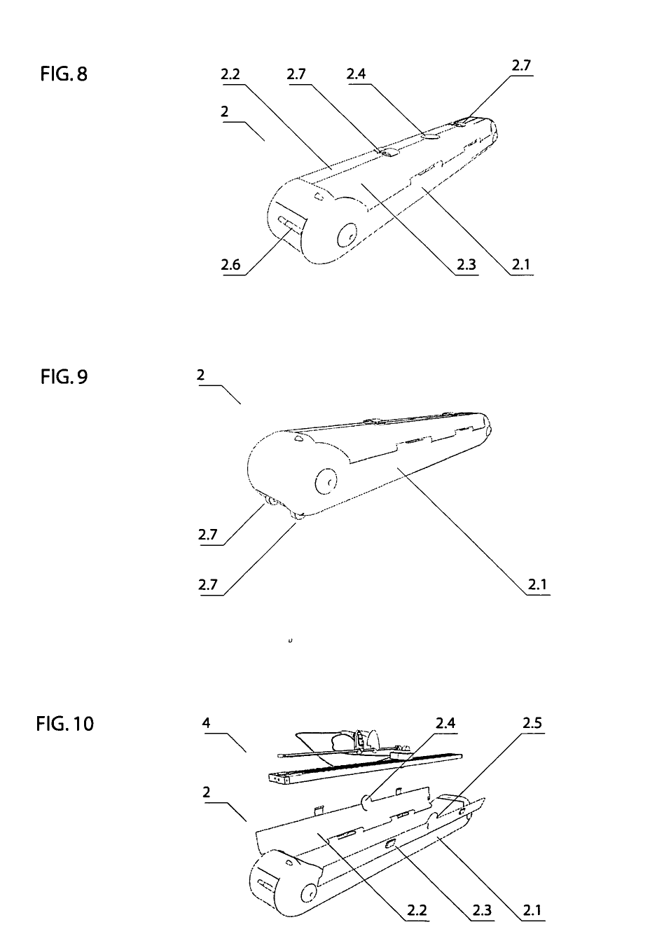

FIG. 8. In such Figure the Protector of the Cartesian Frame in front perspective view is observed, where; the greater drained volume (2.1) in which at the edge of their drained portion, pivoting bodies are connected in its base (2,2 and 2.3). 2 hole-anchorages (2.7) allow to temporarily uniting the 2 pivoting bodies already mentioned. Finally there is a concavity in which a cylinder is connected horizontally (2.6).

FIG. 9. In such Figure the Protector of the Cartesian Frame in rear perspective view is observed, where; in the lower part there is a concavity where a cylinder (2.7) connects in the lateral face.

FIG. 10. In such Figure it is possible to observe a view in front perspective of the Cartesian Protector, in opened stage, in which the folded Cartesian frame is extracted; where the smaller bodies (2, 2 and 2.3) have pivoted for such effect. The semicircular portion (2.4) that projects itself on the second one can be better appreciated (2.5).

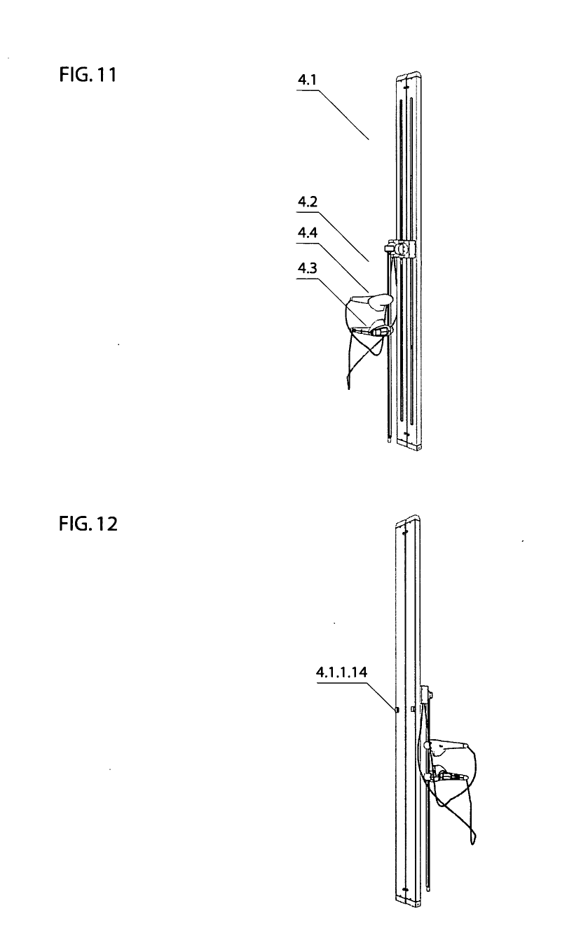

FIG. 11. In such Figure it is possible to observe, in front perspective of the Cartesian Frame, in folded stage; where the tubular Horizontal Axis (4.2) lodges the Master manipulator (4.3) and the slave Manipulator (4.4).

FIG. 12. In such Figure it is possible to observe a rear perspective of the Cartesian Frame, in folded stage, where; the activators (4.1.1.14), that appear from the interior lodged in the middle of each one of these faces referred to in symmetrical form.

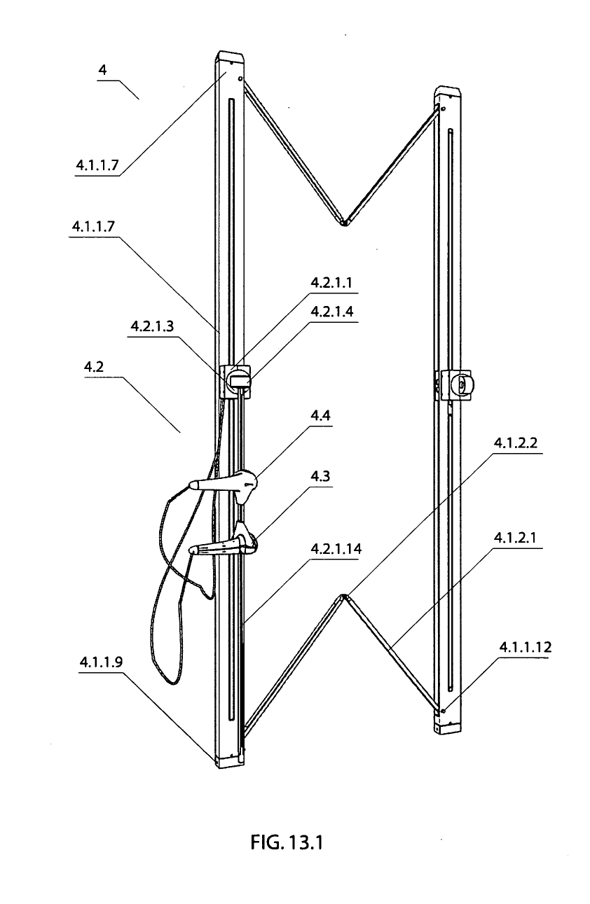

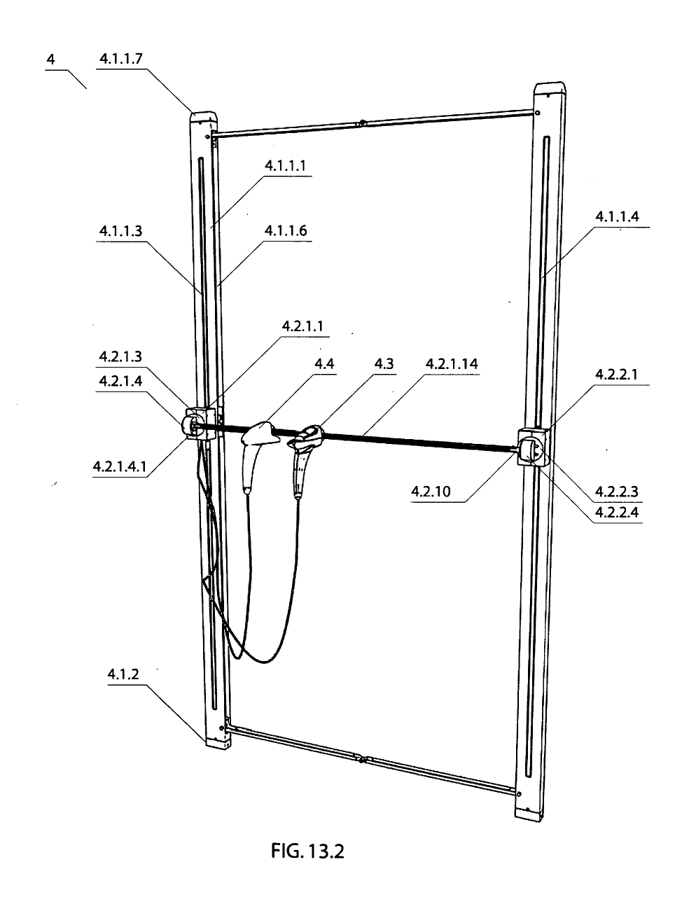

FIG. 13.1. In such Figure a view in front perspective of the Cartesian Frame is observed (4), unfolding, where; the folded laminar body (4.1.2.1) that on its inferior and outer base pivots in connected axis to perforation (4.1.1.12) lodged in drained greater vertical body (4.1.1.2) that allows its possible containment and articulated in its upper and inner end to another smaller laminar body (4.1.2.2)

FIG. 13.2. In such Figure it is possible to observe, a view in front perspective of the Cartesian Frame (4), in unfolded stage, mainly constituted by the Frame (4.1) and Horizontal Axis (4.2), where; the vertical axis (4.1.1.1), drained body, has a slit (4.1.1.3) alongside in its frontal part and another in its internal lateral face (4.1.1.6).

The Horizontal Axis (4.2) is able to slide on the Frame (4.1) in its greater axial direction. The drained polyhedral volume (4.2.11) incorporates the contiguous cylindrical volume (4.2.1.3) allowing its axial spin, in turn, this incorporates the next drained semi cylindrical volume (4.2.1.4). This last one receives a laminar body in its internal lateral face (4.2.1.14), by means of a cylindrical piece that connects each other (4.2.1.4.1). In an opposite end the drained polyhedral volume (4.2.2.1) that incorporates contiguous cylindrical volume (4.2.2.3) on which the following drained semi cylindrical volume horizontally slides (4.2.2.4) so as to secure the pivoting position of the assembly for its final stage of spreading out, is projected. The laminar body (4.2.1.14) allows that both the Master Manipulator (4.3) and the Slaved Manipulator (4.4) slide themselves in their greater axial direction.

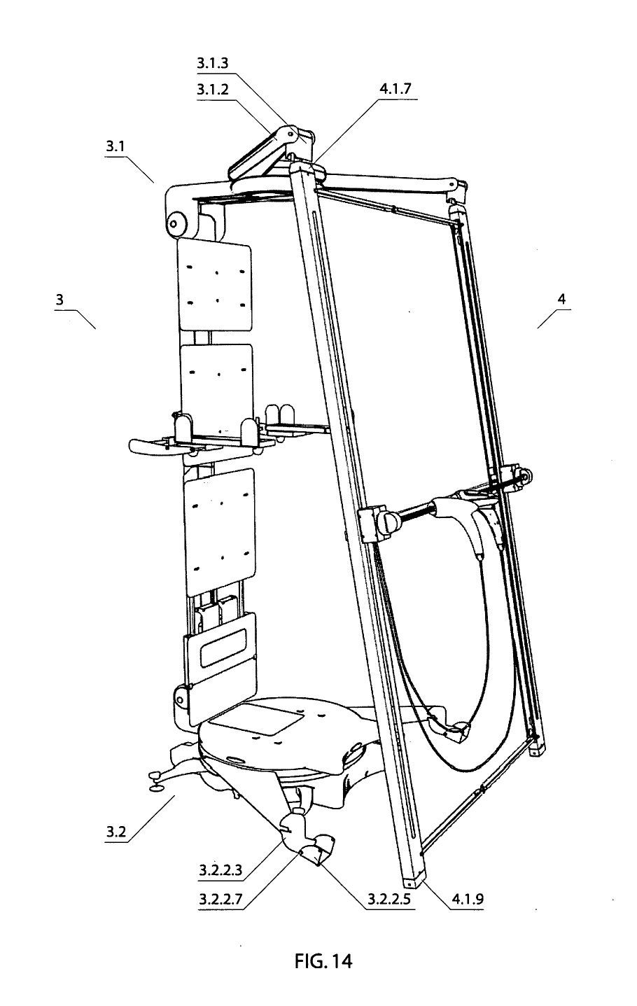

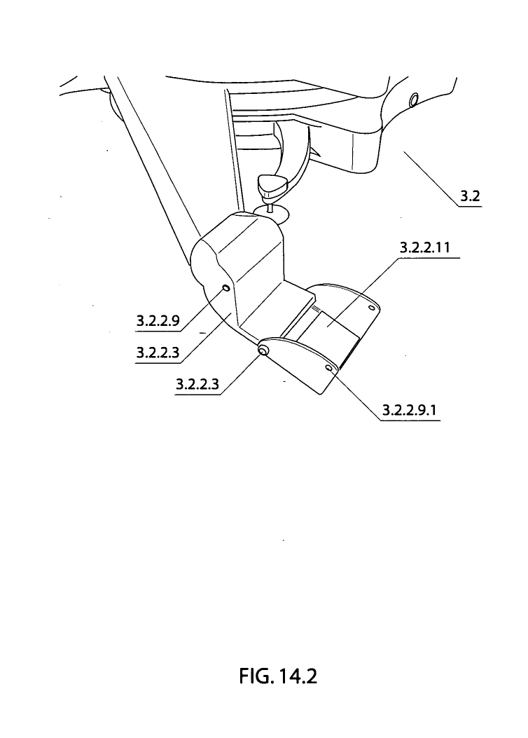

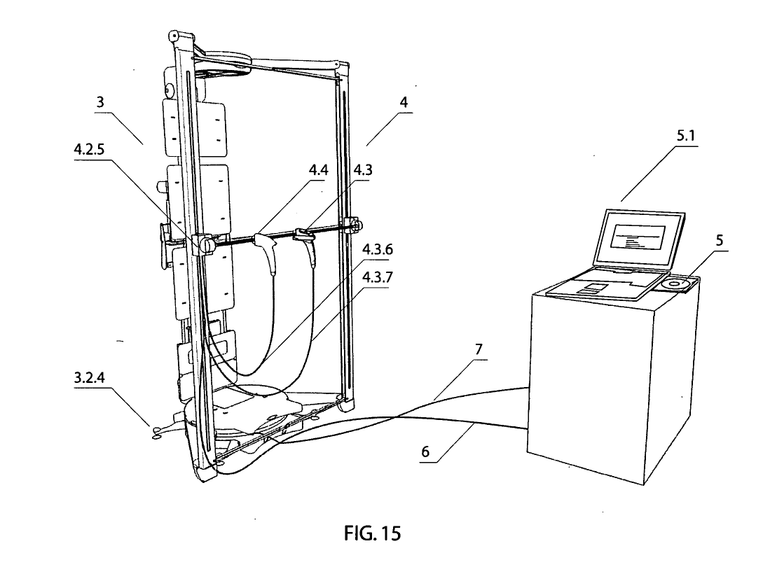

FIG. 14. In such Figure it is possible to observe the view in front perspective of the Postural Rectifier (3) and the Cartesian Frame (4), in coupling phase. Operation conducted by an individual for which 2 sequential actions are performed. First, the sharp truncated body (4.1.7) belonging to (4) must be directed towards and in direction of the interior of the laminar body, Upper Receptor (3.1.3) pivoting in (3.1.2). Second, the lower end, where the drained body (4.1.9) must be directed towards and in direction of the body (3.2.2.3) to be then secured by means of a laminar body (3.2.2.5) pivoting to (3.2.2.7) located in the body already mentioned (3.2.2.3) that has an automatic insuring internal system.

FIG. 14.2. In such Figure, detail in front perspective view of the Receptor, is observed, where; a thin layer of determined thickness is projected (3.2.2.5.1) in the internal face of (3.2.2.5). The perforation (3.2.2.9) positioned and next to the edge in the lateral face of (3.2.2.5) that coincides in its closing phase with the body comes into view (3.2.2.9.1) from the interior of (3.2.2.3).

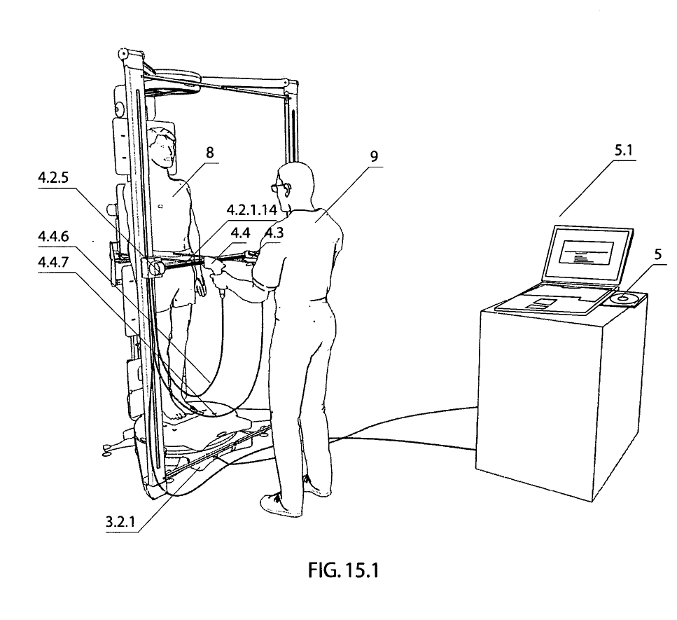

FIG. 15. In such Figure it is possible to observe a view in front perspective of the Postural Rectifier (3), Cartesian Frame (4), complementary units coupled and a standard Notebook (5.1) in which the Software (5) of certain characteristics is noticed, in phase of data transfer and leveling. Where the latter after settling and setting up, both supplies power and; records, stores, processes and post processes the anthropometric data that are transferred from the units (4 and 3). At first instance the Manipulators deliver the information to the drained polyhedral body (4.2.5) that contains circuits by means of suitable wires; (4.3.7) for the Master Manipulator (4.3) and (4.4.6) for the Slave Manipulator (4.4). Finally from the drained polyhedral body (4.2.5) the suitable interface wire is connected (6) and from the base (3.2.1) similarly (7).

FIG. 15.1. In such Figure it is possible to observe a view in front perspective of the Postural Rectifier (3), Cartesian Frame (4), complementary units coupled and a standard notebook (5.1) in which the software (5) of certain characteristics is noticed, in phase of data transfer and leveling, that in addition shows the users utilizing the system, where the user to measure (8) is in position of anthropometric attention type (standing up), received by the Rectifier Postural already mentioned (3) and the user who performs the measurement (9) manipulating the Slave Manipulator (4.4) and the Master Manipulator (4.3) lodged in Cartesian Frame (4).

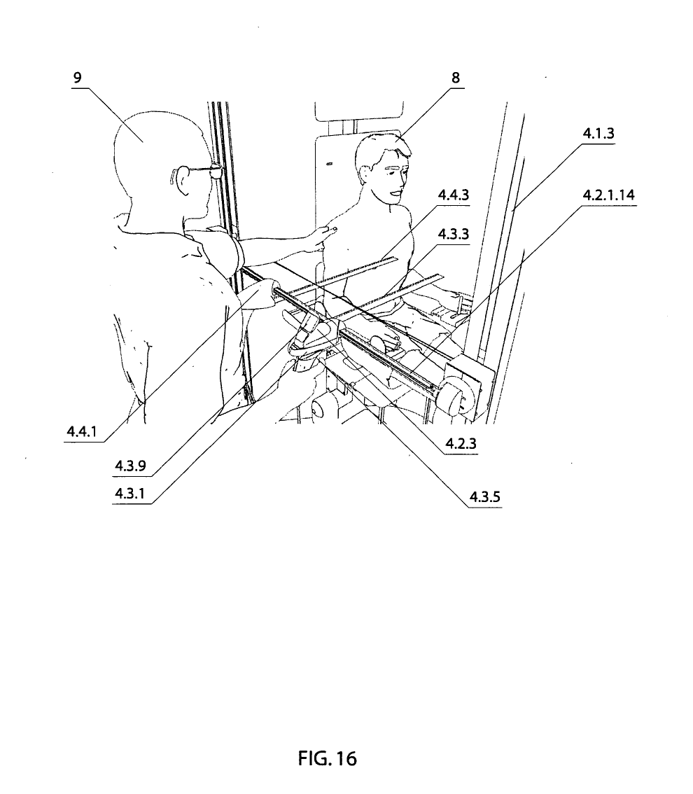

FIG. 16. In such Figure the illustrative detail of the view in front perspective is observed, corresponding to the standard measurement procedure that is performed to the measured user (8) by the professional who studies anthropometry, measurer user (9), where; the Horizontal Axis (4.2), Slave Manipulator (4.4) is horizontally movable, from whose drained greater body, casing (4.4.1), from its frontal part emerges a visible light beam (4.4.3) that projects perpendicularly to the horizontal Axis (4.2). The Master Manipulator (4.3), thus symmetrically to the previous one, in addition counts on a pivoting digital display of metrological information (4.3.9) lodged in the upper part of the drained greater body (4.3.1) that will eventually record by means of tactile pulsing, the hand previously grasped, of a controller (4.3.5).

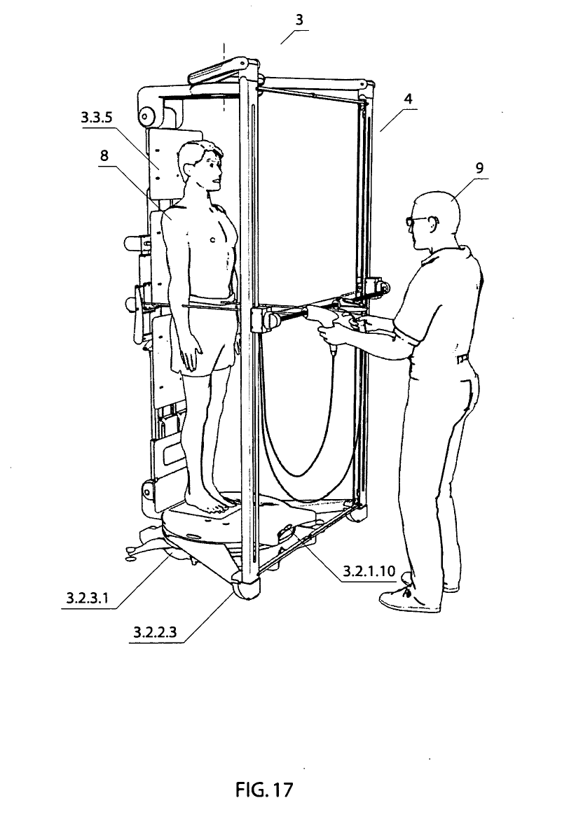

FIG. 17. In such Figure a view in frontal perspective is observed, in which a standard measurement is performed, where the Cartesian Frame (4) is rotated in Frontal View Position, parallel to flatness of (3.3.5) received by receptors (3.2.2.3), distant and coaxial to (3.2). Positionally secured by activating (3.2.1.10).

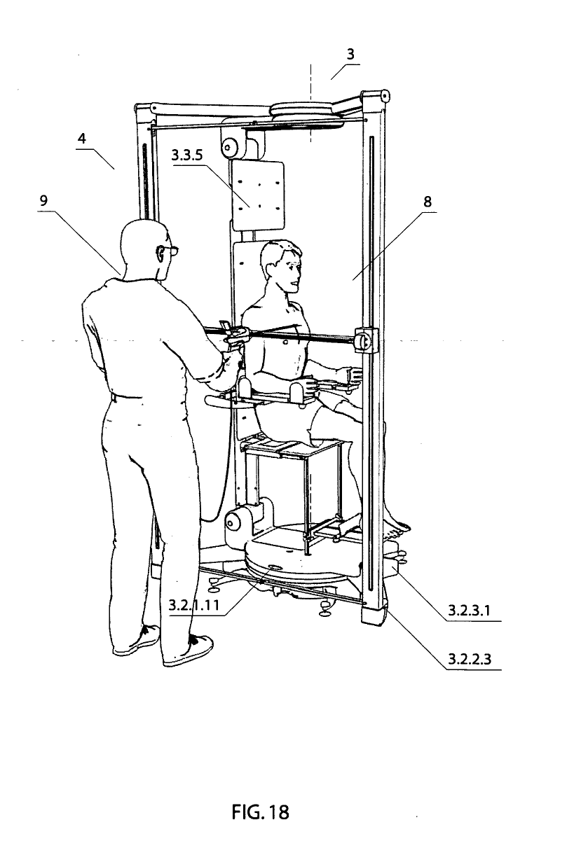

FIG. 18. In such Figure a view in frontal perspective is observed, where the Cartesian Frame (4) turned 90° right with respect to flatness of (3.3.5) received by receptors (3.2.2.3), distant and coaxial to (3.2.3.1). Positionally secured by activating (3.2.1.11).

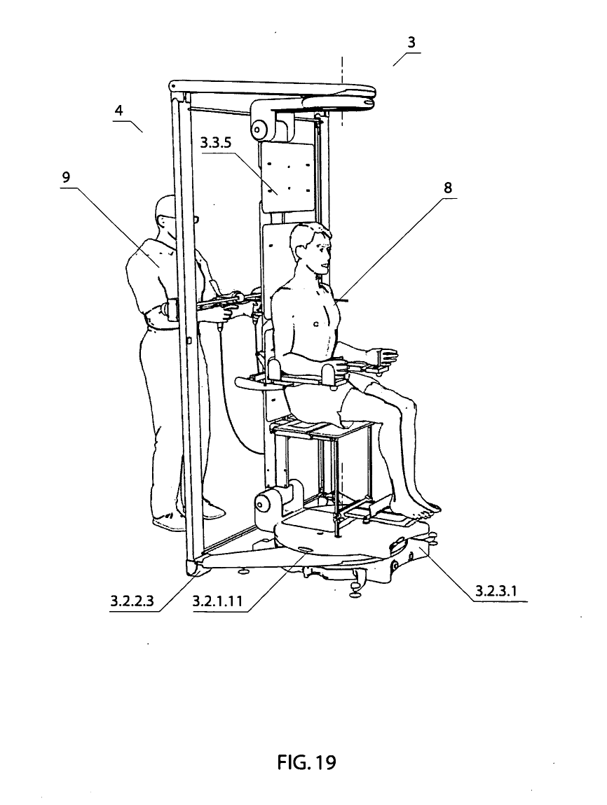

FIG. 19. In such Figure a view in frontal perspective is observed, the Cartesian Frame (4) turned 180° right with respect to flatness of (3.3.5) received by receptors (3.2.2.3), distant and coaxial to (3.2.3.1). Positionally secured by symmetric activating to (3.2.1.11).

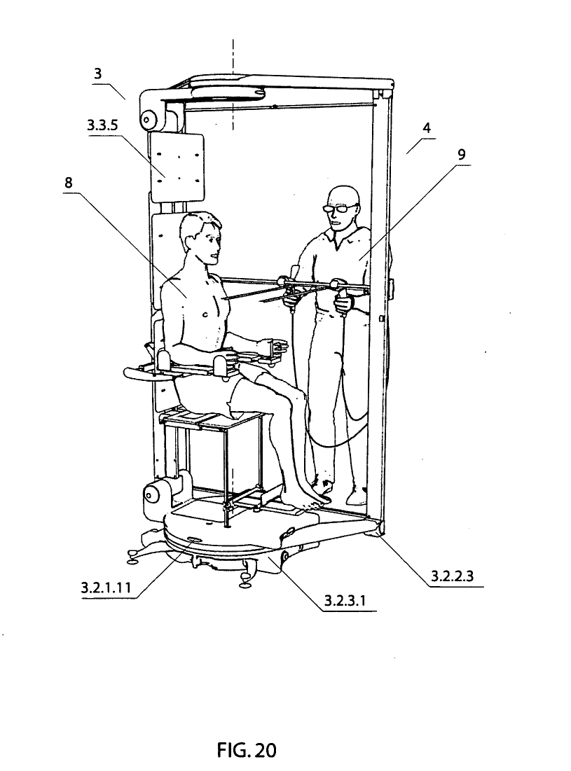

FIG. 20. In such Figure a view in frontal perspective is observed, the Cartesian Frame (4) turned 270° right with respect to flatness of (3.3.5) received by receptors (3.2.2.3), distant and coaxial to (3.2.3.1). Positionally secured by symmetric activating to (3.2.1.11).

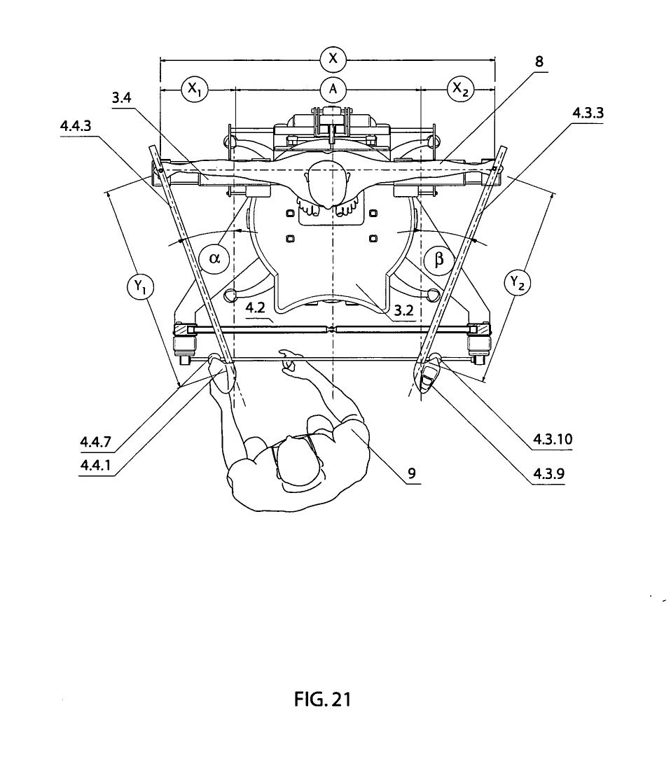

FIG. 21. In such Figure a view in upper cut is observed, in which a Horizontal Oblique Measurement is performed, where; the Slave Manipulator (4.4.1) is able to pivot in rotular body (4.4.7) in which the visible luminous beam (4.4.3) conceives an Alpha angle with respect to the axis of sagittal symmetry of the unit. Symmetrically, the Master Manipulator (4.3.9) pivots in (4.3.10) also projecting a visible luminous beam (4.3.3) forming a Beta angle with respect to the axis of sagittal symmetry of the unit.

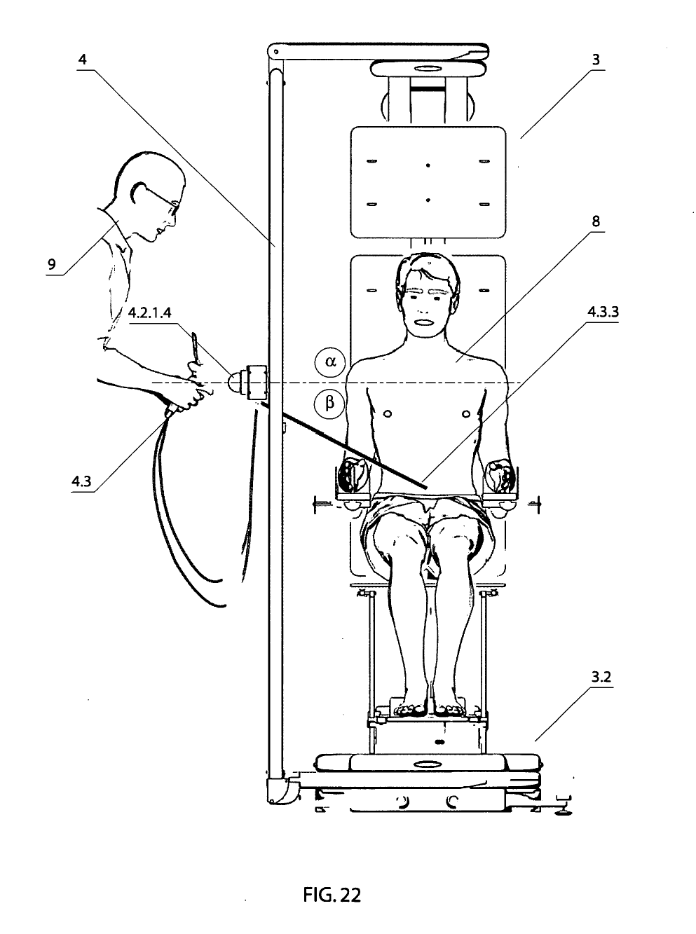

FIG. 22. In such Figure a frontal orthogonal view is observed, where the Cartesian Frame is rotated in sagittal position, in which a Vertical Oblique Measurement is performed, where; the Cartesian Manipulators, rotate around (4.2.1.4) forming a positive Alpha angle and a negative Beta angle for the visible luminous beam (4.3.3) with respect to a horizontal axis.

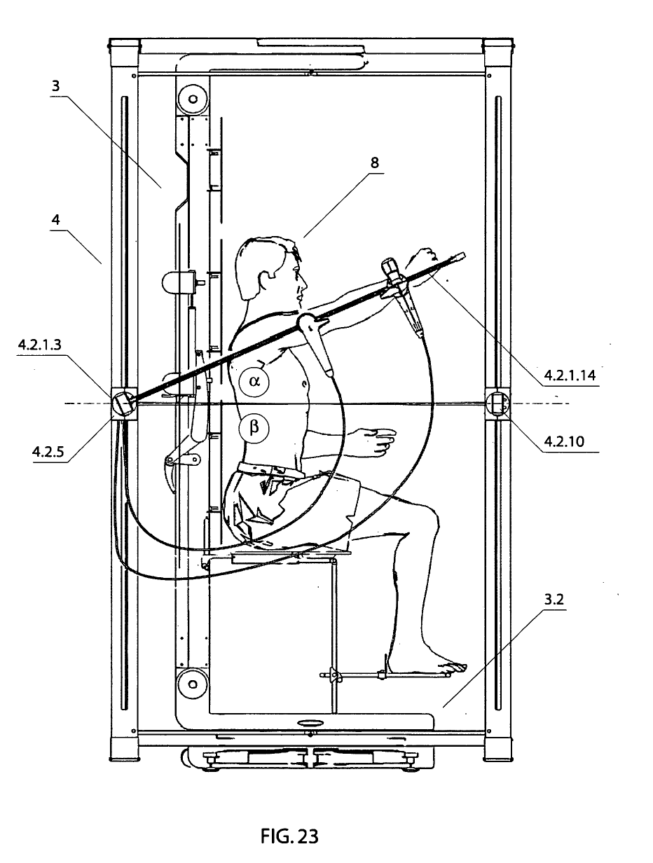

FIG. 23. In such Figure a lateral orthogonal view is observed, where the Cartesian Frame is rotated in sagittal position, in which a Horizontal Aligned Measurement is performed, where; the laminar body (4.2.1.14), in which the Cartesian Manipulators are positioned, is able to coaxially pivot the drained cylindrical body (4.2.1.3) forming the positive Alpha and negative Beta angles.

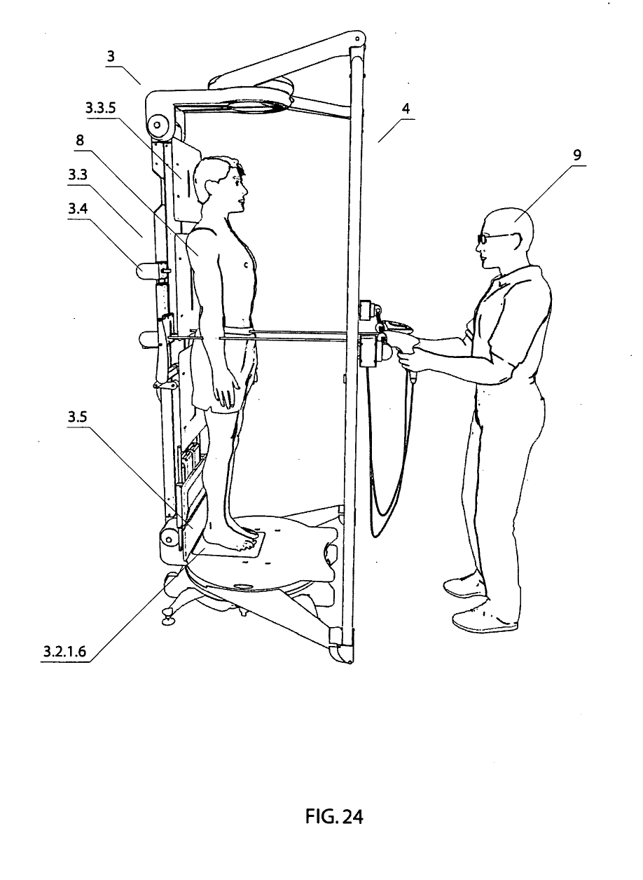

FIG. 24. In such Figure a view in lateral perspective is observed, in which a standard measurement is performed, arranged to receive the standing up position, where; the measured user (8) in protocol standing up posture is supported by flatness of column (3.3) and received by backed and folded seat (3.5). In addition a rectangular body (3.2.1.6) embedded in the upper face is noticed in (3.2)

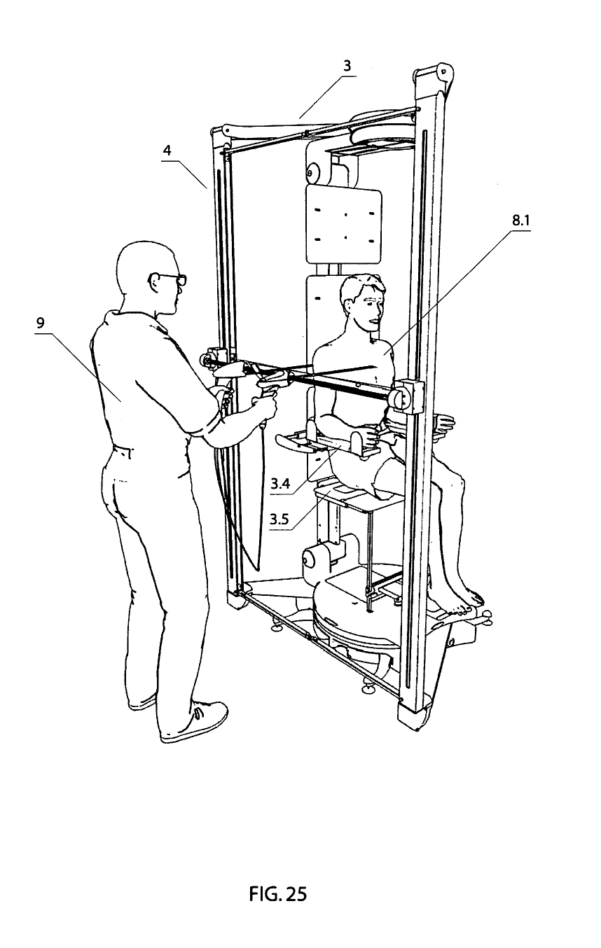

FIG. 25. In such Figure a view in lateral perspective is observed, in which a standard measurement is performed, arranged to receive the sedent (in sitting position) posture; where the measured user in sedent (in sitting position) posture (8.1), is received by the armrest (3.4) and the seat (3.5), which extend, rotate, depress and adjust.

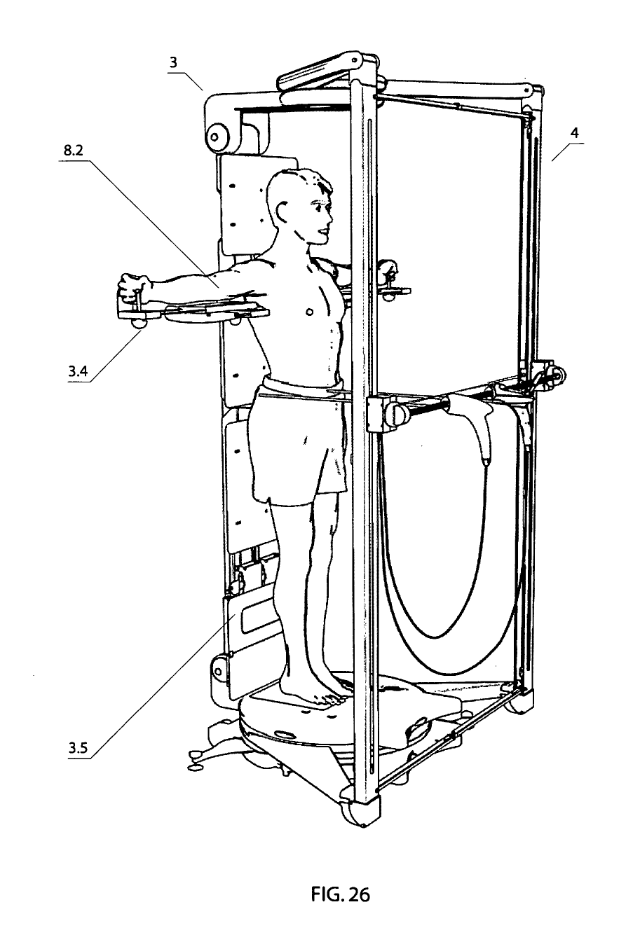

FIG. 26. In such Figure a view in lateral perspective is observed, in which a standard measurement is performed, arranged to receive the arm span posture; where this measured user (8.2) in arm span position (8.1), is received by the armrest (3.4) and the seat (3.5), which extend, rotate, depress and adjust.

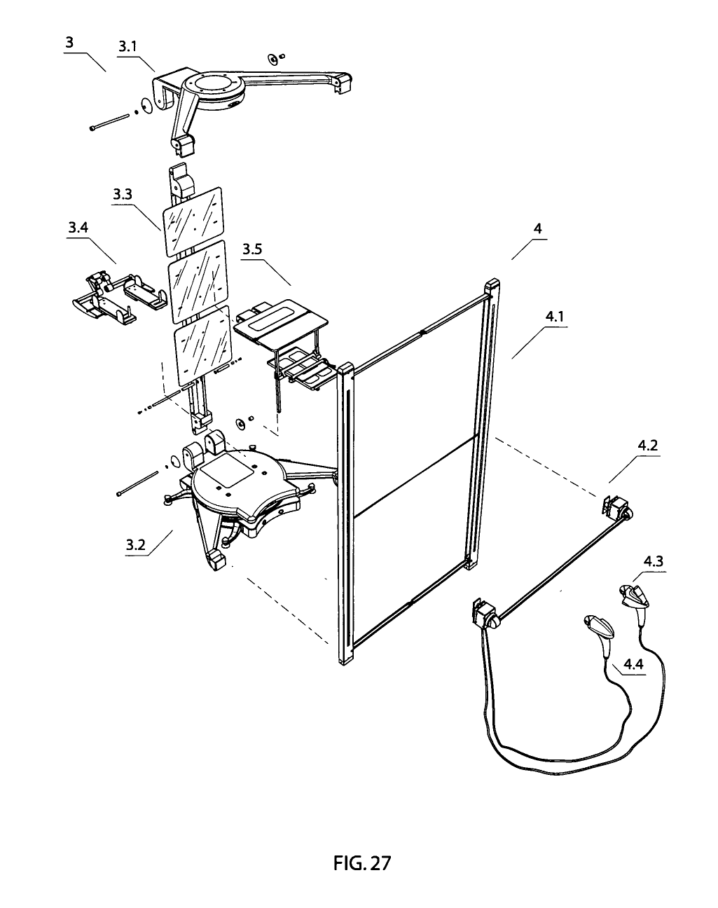

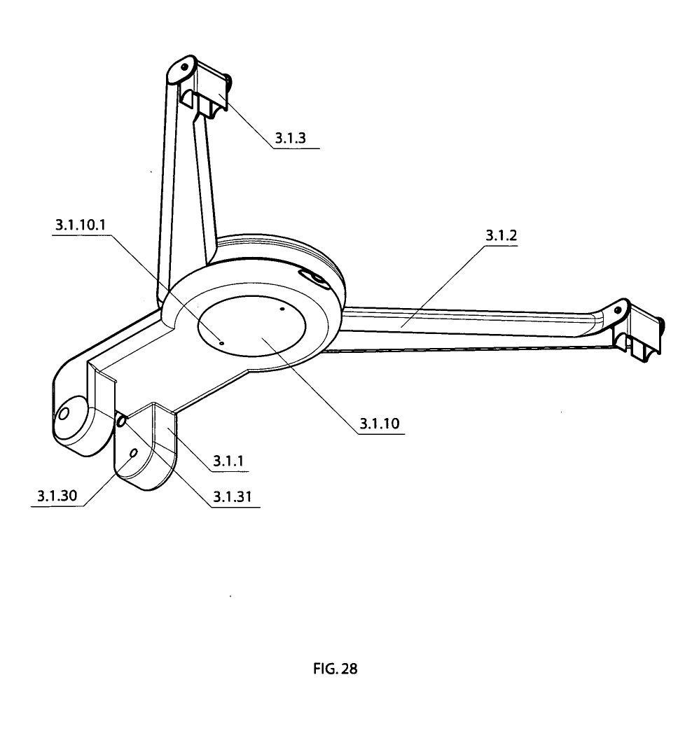

FIG. 27. In such Figure a view in front perspective of quartering of the main components by way of quartering is observed.

FIG. 28. In such Figure a view in upper front perspective of the upper support assembly is observed (3.1), where; the Receptor Frame B (3.1.2) corresponds to an initially cylindrical body that tangentially projects an extension that in its terminal part a smaller polyhedral body is removed. In the inner faces of this drained portion a smaller laminar body lodges perpendicular to the axis of the Receptor extension A (3.1.3) already mentioned, which connect by means of a cylindrical body Receptor Connector (3.1.18) that allows a pendular movement of (3.1.3).

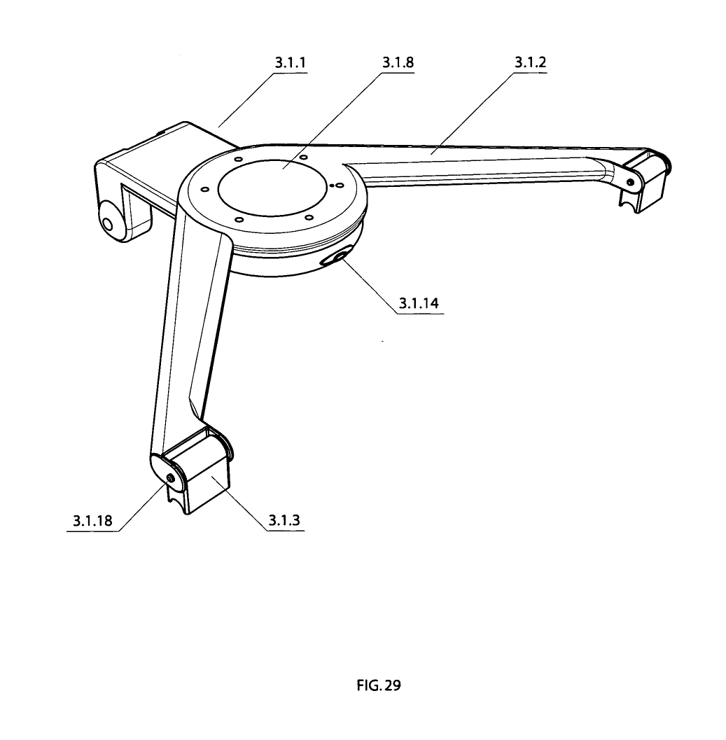

FIG. 29. In such Figure a view in lower front perspective of the upper support assembly is observed (3.1), where; the greater body (3.1.1) has a polyhedral portion in its lower rear part that projects from its external lateral edge with determined dimension, where its completion is achieved by forming a semi cylinder, in whose capable center totally passes a Perforation (3.1.30), in addition a Perforation B of certain depth lodges near the previous. This defined body is symmetrically constructed to the longitudinal vertical plane to (3.1.1).

The body (3.1.10) in its external part has 2 perforations (3.1.10.1) equidistant of the capable center.

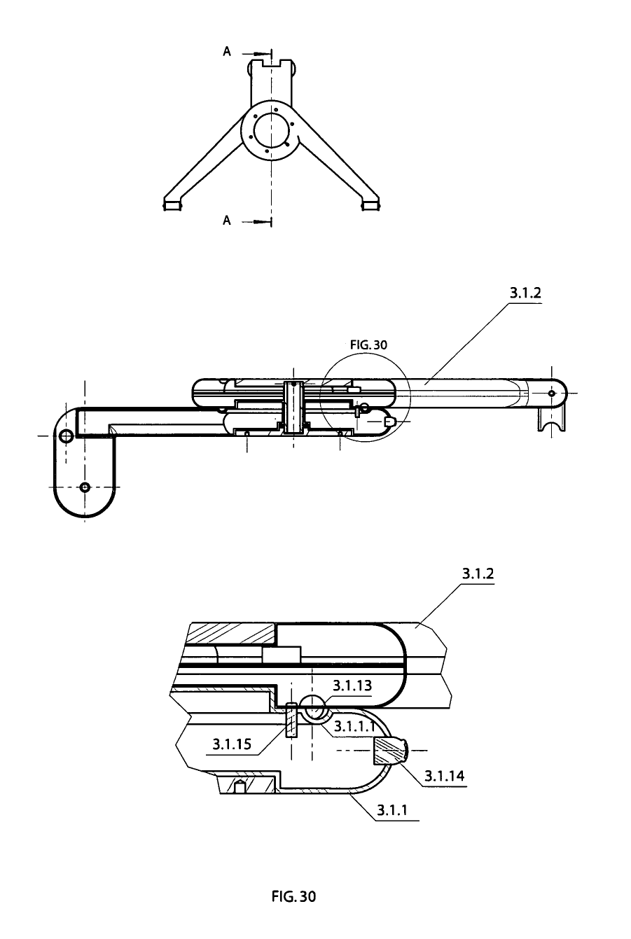

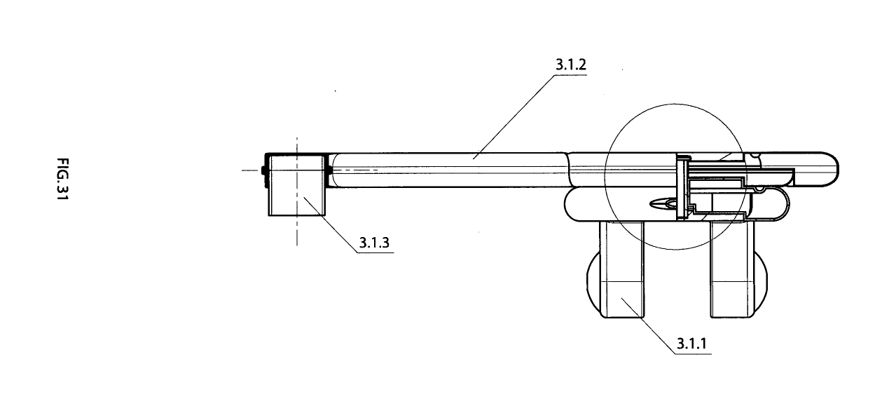

FIG. 30. In such Figure a detail of the upper view A cut of the upper support assembly is observed (3.1), where; the base Support (3.1.1) has a continuous slit (3.1.1.1) of semicircular section that lodges a spherical body (3.1.1.3) of certain dimensions and properties that is fitted in a semispherical slit in the inner face of (3.1.2) equidistantly distributed and at a certain distance of the capable circumcenter. It is also possible to notice a Safe cylindrical element (3.1.15) that is adjusted in a capable perforation in the drained body (3.1.2), perpendicularly passing, thence in addition, allowed by its counterform in (3.1.1). From the latter, in addition, it comes into view, from its drained interior and by means of an opening in its frontal part, a truncated ellipsoid body, Button (3.1.14).

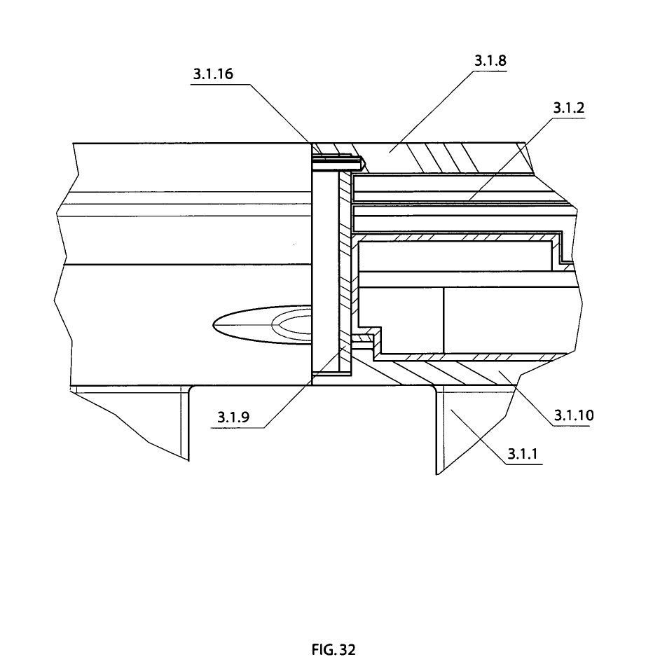

FIG. 32. In such Figure a detail of the FIG. 31 is observed, broken view in frontal view of the upper support assembly (3.1), where; the drained greater body base Support (3.1.1) receives, in a perforation of certain diameter with center in the origin of its greater curvature, coaxially to, Frame Receptor B (3.1.2) closed in its lower part by a cylindrical body of certain thickness Receptor Axis A2 (3.1.10) and in its upper part by a cylindrical body of certain thickness and diameter, Receptor Axis A (3.1.8), these latter share extreme faces with the outer surfaces in coplanar form to Frame Receptor B (3.1.2). In addition, a truncated cone laminar body (3.1.16) passes through the bodies (3.1.8) and (3.1.9).

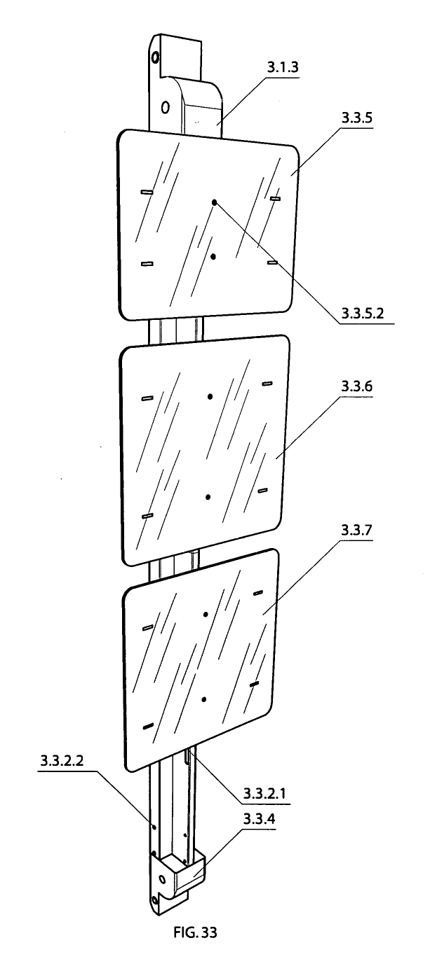

FIG. 33. In such Figure a view in front perspective of the Column assembly is observed (3.3), where; an extended body (3.3.2.1) is removed from the drained body (3.3.2.2).

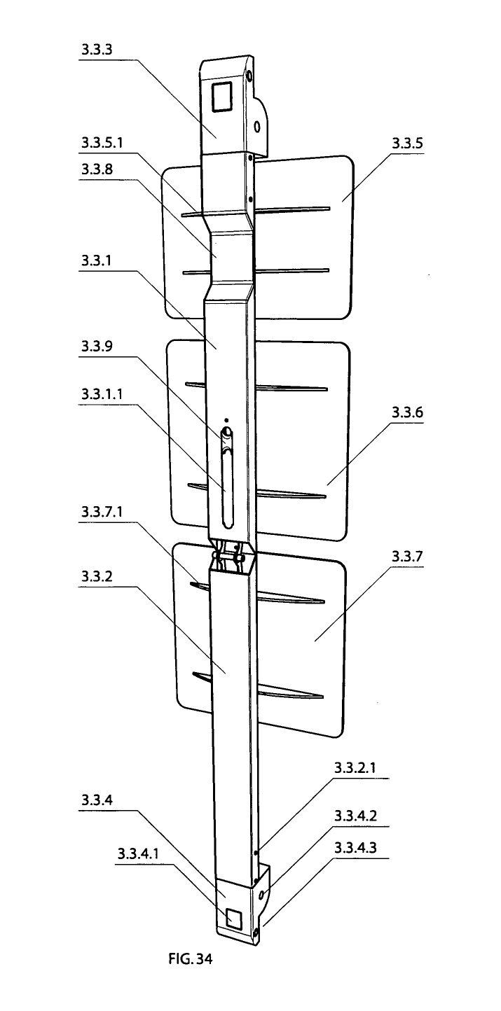

FIG. 34. In such Figure a view in rear perspective of the Column assembly is observed (3.3), where; a drained body (3.3.2) is connected to a drained polyhedral body that has a semi cylindrical portion (3.3.4) by means of spiral connectors (3.3.2.1). The body (3.3.4) has the subtraction of a polyhedral body, inside and from there a body with such form is projected tangential to the outer face (3.3.4.1) tolerant for its movement in its projected axis; similarly a cylinder (3.3.4.3), this, if, in both lateral faces and approximated to the lowest face and a passing perforation to the body, in greater circumcentre of the lateral face. A laminar body (3.3.7) of certain thickness and rectangular base that attaches to the frontal face of (3.3.2), is also distinguished, from whose joint another laminar body is incorporated (3.3.7.1). This attachment is achieved due to cylindrical and spiral bodies (3.3.5.2.) perpendicular to the greater axis of the assembly (see FIG. 33).

It is possible to notice, the subtraction of a body (3.3.1.1) located in the rear face of the drained body (3.3.1) which contains, and comes into view from the interior, a body (3.3.9) that in its upper and lower faces have semi cylindrical recesses, able to slide in the already mentioned one. This drained body (3.3.1), in addition has a considerable depression (3.3.8) approximated to its upper extreme edge.

These drained greater bodies (3.3.1 and 3.3.2) connect by means of 2 laminar bodies (3.3.10 and their symmetrical one) to both internal sides which have a perforation that in turn, is finally connected by a cylindrical body (3.3.11).

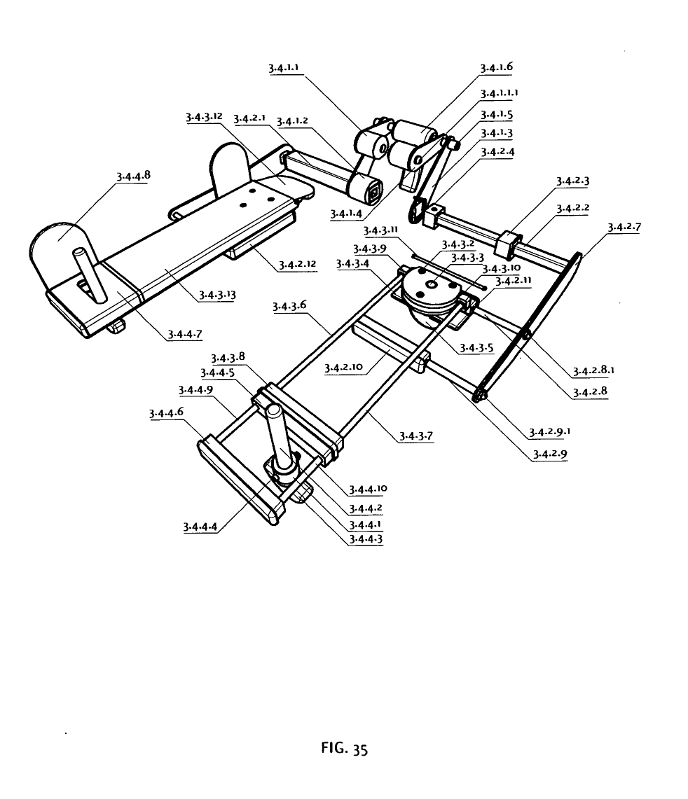

FIG. 35. In such Figure a view in front perspective of the Armrest assembly is observed (3.4), where; a cylindrical body (3.4.1.1) is connected by means of a cylindrical body of smaller diameter (,1 3.4.1.1) that passes through this one and a decreasing laminar body (3.4.1.4). This latter, in turn, in its other end is connected to a cylindrical body (3.4.1.5), that crosses, by means of an eccentric perforation, a cylindrical body (3.4.1.6) from which a decreasing body of curved axis with its center displaced to the interior of the assembly is projected in its inferior average portion.

United to (3.4.1.3) in its outer face a drained polyhedron is projected (3.4.2.1), in whose interior another similar body of smaller dimension lodges coaxially (3.4.2.2) that is connected to a body that fills this inner space (3.4.2.4) connected, in turn, by means of a cylindrical body (3.4.2.5). To the body (3.4.2.1) at its external edge a body that fills the distance between this one and (3.4.2.2), body (3.4.2.3) is attached. A greater laminar body (3.4.2.7) is joined in its extreme edge to the body (3.4.2.2) perpendicular to this one and horizontal to the assembly from which two cylindrical bodies (3.4.2.9,1) and (3.4.2.8,1) pass by means of aligned perforations that distance the bodies (3.4.2.10) and (3.4.2.11) in whose substitute space two cylindrical tubular bodies are integrated (3.4.2.9) and (3.4.2.8). The bodies (3.4.2.10) and (3.4.2.11) are, by construction, aligned and distant, which are joined by a polyhedral laminar body (3.4.2.12), that in its lower face has a slit of certain dimensions that vertically receives a body of cylindrical base that projects downwards by means of a significantly semi cylindrical greater body (3.4.3.5). On this one, it projects a cylindrical body (3.4.3.3), which is wedged by another cylinder (3.4.3.1) with a significantly greater diameter, that in its rear part has a cut and in its upper face three equidistant perforations to its common axial. In these perforations three cylindrical bodies are lodged (3.4.3.2) that allow connecting a greater laminar body (3.4.3.13), from quite near to its rear edge. In the opening of its other end, a polyhedral body (3.4.3.8) comes into view from its interior. In the internal face of this one, 2 cylindrical bodies are projected (3.4.3.6) and (3.4.3.7) in whose ending are two smaller polyhedral bodies (3.4.3.9) and (3.4.3.10).

From the cylindrical bodies (3.4.3.6) and (3.4.3.7), in their terminal and outer face, a polyhedral body (3.4.4.6) and to certain distance towards the interior of the assembly, a body (3.4.4.5) that has for such position 2 perforations that let pass such cylindrical bodies (3.4.3.6) and (3.4.3.7) are connected. A laminar body (3.4.4.7) covers these latter, which in whose upper and lower face has a slit that lodges a body that projects itself upwards; this one is cylindrical (3.4.4.10). Connected to this latter and lodged inside (3.4.4.7) is a tubular body (3.4.4.1) in which a cylinder (3.4.4.4) of smaller diameter crosses. On the base of (3.4.4.10) a body (3.4.4.3) similar to (3.4.3.5) is connected by means of an inner thread.

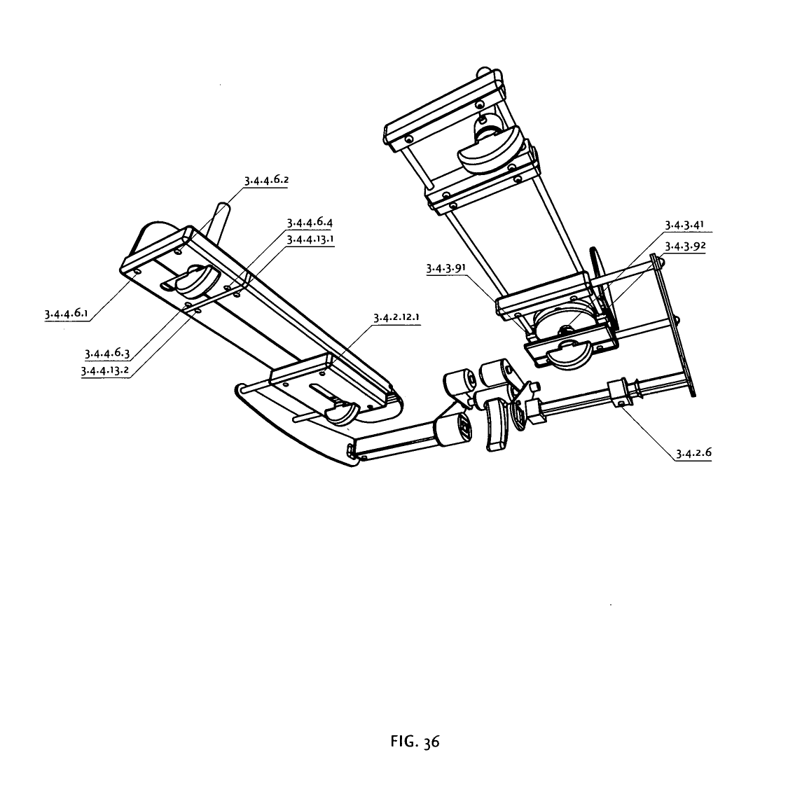

FIG. 36. In such Figure a view in lower perspective of the assembly Armrest (3.4) is observed, where the spiral cylindrical bodies can be noticed (3.4.4.6, 1), (3.4.4.6, 2) (3.4.4.6, 3) and (3.4.4.6, 4) that position (3.4.4.7). Similarly, (3.4.3.13, 1) and (3.4.3.13, 2) that position (3.4.3.13).

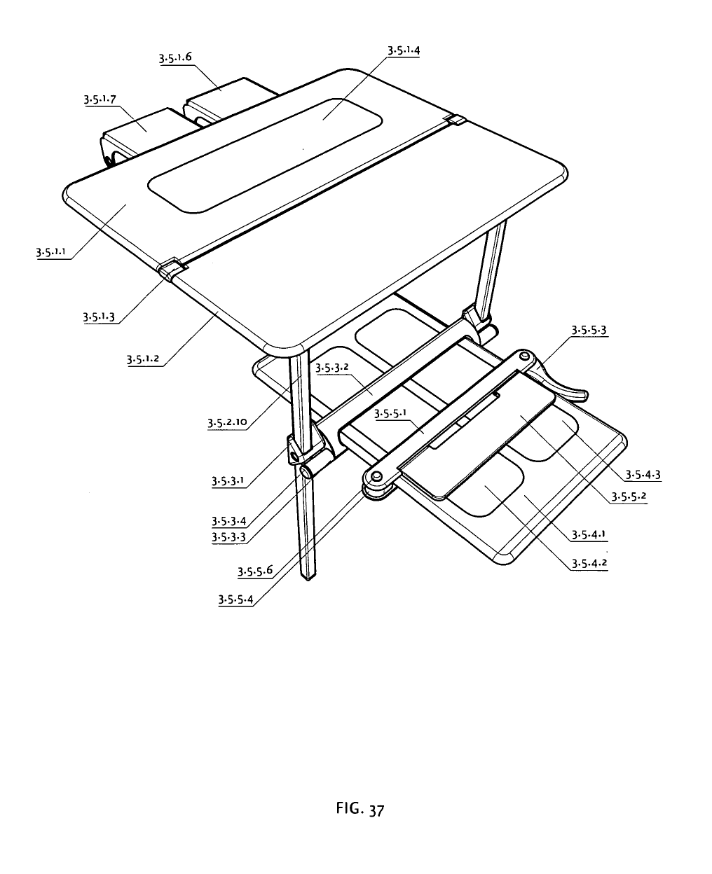

FIG. 37. In such Figure a view in front perspective of the Seat Assembly is observed (3.5), where; two laminar greater bodies of considerable thickness (3.5.1.1) and (3.5.1.2) are united by a smaller body (3.5.1.3). An embedded rectangular laminar body (3.5.1.4) approximately at the center of (3.5.1.1). Another greater laminar body (3.5.4.1) is introduced in an extended body of elliptical section (3.5.3.2) from whose extreme face a body of triangular section is connected (3.5.3.1) which is vertically crossed by a square tubular body (3.5.2.10). On the outer lateral face of (3.5.3.2), a cylindrical body is also projected (3.5.3.3) finished at its end, and jointly, a significantly smaller cylindrical body (3.5.3.4).

An extended body (3.5.5.6) of rectangular base is also observed, from whose ends a semi cylinder is formed, cross-sectionally arranged on the upper face of (3.5.4.1), this one has a cylindrical body (3.5.5.6) that crosses it in its capable center, to both ends, in the right end, in the lower face of such portion, a body (3.5.5.4) is projected, and in the left end, also in the lower portion, a body (3.5.5.3) is projected whose axis has double curvature, those of the second and most external one, its radial center is towards the outside of the assembly. On the upper face of (3.5.4.1) two parallel rectangular bodies are embedded (3.5.4.2) and (3.5.4.3). In addition, a laminar body (3.5.5.2) of certain dimensions is slightly inserted to the body (3.5.5.1).

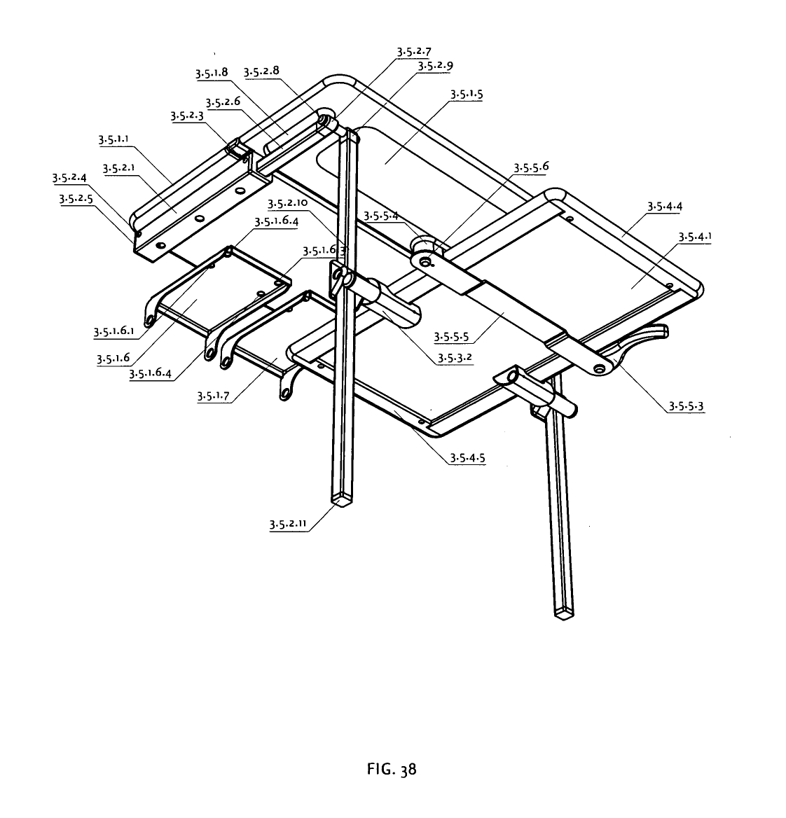

FIG. 38. In such Figure, a view in lower perspective of the Seat assembly is observed (3.5), where; in the lower and rear face of the body (3.5.1.1) parallel to the lateral edge a polyhedral tubular body is projected (3.5.2.1), from whose interior, and subsequently a body is projected (3.5.2.5), now in its frontal opening a polyhedral tubular body (3.5.2.2) is projected from where another polyhedral tubular body (3.5.2.6) of dimensionally smaller section emerges from, this in its frontal end is solidly joined to a body of semi cylindrical ending (3.5.2.7) which in its outer lateral face is crossed by means of a cylindrical body (3.5.2.8) that joins it to a similar distant body (3.5.2.9). From this last body downwards a polyhedral tubular body (3.5.2.10) of considerable dimension in its vertical axis is projected, that in its extreme low part a smaller body is united (3.5.2.11).

It addition, in the lower part of (3.5.1.1) a laminar body (3.5.1.6) is observed, connected by means of cylindrical bodies (3.5.1.6,1), (3.5.1.6,2), (3.5.1.6,3) and (3.5.1.6,4) in its frontal portion, external rear portion to the rear edge of (al). We can also see that the body (3.5.4.1) is closed at its ends by (3.5.4.4) and (3.5.4.5). And a nonflat laminar body (3.5.5.5) that joins (3.5.5.4) and (3.5.5.3).

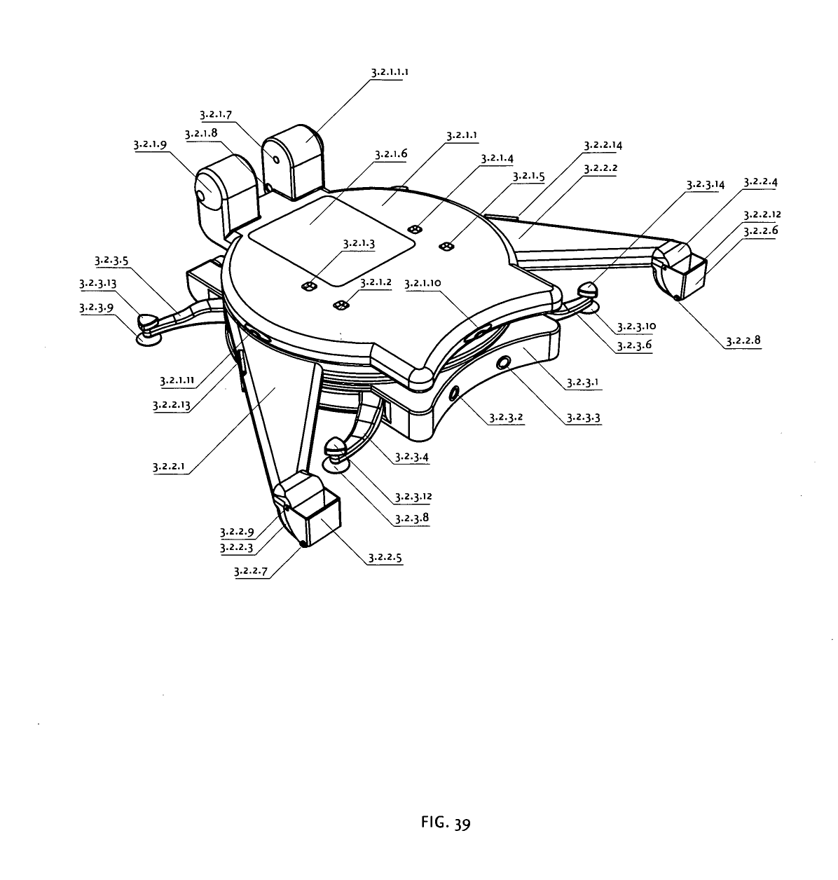

FIG. 39. In such Figure a view in front perspective of the Lower Support assembly is observed (3.2), where; a drained body (3.2.1.1) of mainly circular base and that both in its frontal part and rear part presents a rectangular prolongation of certain height (3.2.1.1), in its rear surface two parallel bodies (3.2.1.1,1) are projected and in its frontal portion a concavity is observed. Such a body (3.2.1.1,1) has in its inner lateral face a perforation (3.2.1.7) of certain depth, and over this one a perforation that passes trough it (3.2.1.8). Four square-based depressions (3.2.1.2), (3.2.1.3), (3.2.1.4) and (3.2.1.5) lodged on the upper face, two of which are longitudinally aligned and approximate to the outer edge, while the other two are symmetrical to these in a longitudinal plane. In the frontal concavity we can see an ellipsoid body (3.2.1.10). In the convex face we can see similar body (3.2.1.11).

Under (3.2.1.1) we can observe, a body (3.2.2.1) that extends tangentially to cilindricity of (3.2.1.1) which decreases and that in its extreme portion is united to a body (3.2.2.3) that projects itself downwards frontally held, by a laminar body (3.2.2.5) that in its lower lateral base has a cylindrical body of reduced diameter (3.2.2.7), on this same face a perforation (3.2.2.9)

Again, under (3.2.2.1) we can see a drained body (3.2.3.1), of similar base to (3.2.1.1), from whose frontal part also concave are two slits, (3.2.3.2) and (3.2.3.3). From its lateral face we can observe a greater slit, from which a decreasing body (3.2.3.4) of curved axis emerges, whose radius center is towards the interior of the assembly. Terminal to such body and on its upper face is a body of triangular base of convex sides (3.2.3.12), and at the other side of this one a truncated cone body (3.2.3.8).

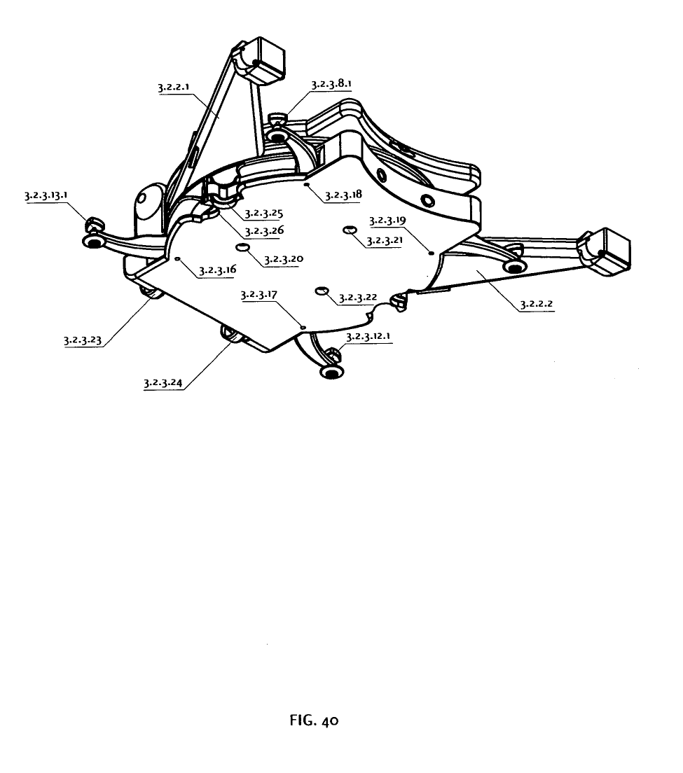

FIG. 40. In such Figure a view in lower perspective of the Lower Support assembly is observed (3.2), where; in the lateral and lower face of the body (3.2.3.1), a slit (3.2.3.25) and another symmetrical one (3.2.3.26); we can also see three greater perforations (3.2.3.20), (3.2.3.21) and (3.2.3.22) equidistant to the capable common axis; in addition two smaller perforations (3.2.3.16) and (3.2.3.18) that are symmetrically repeated (3.2.3.17) and (3.2.3.19) to the longitudinal plane. In the rear portion we can see a removed rectangular volume, from whose lateral internal face a cylindrical body emerges (3.2.3.24) which is symmetrically repeated (3.2.3.23) to such level.

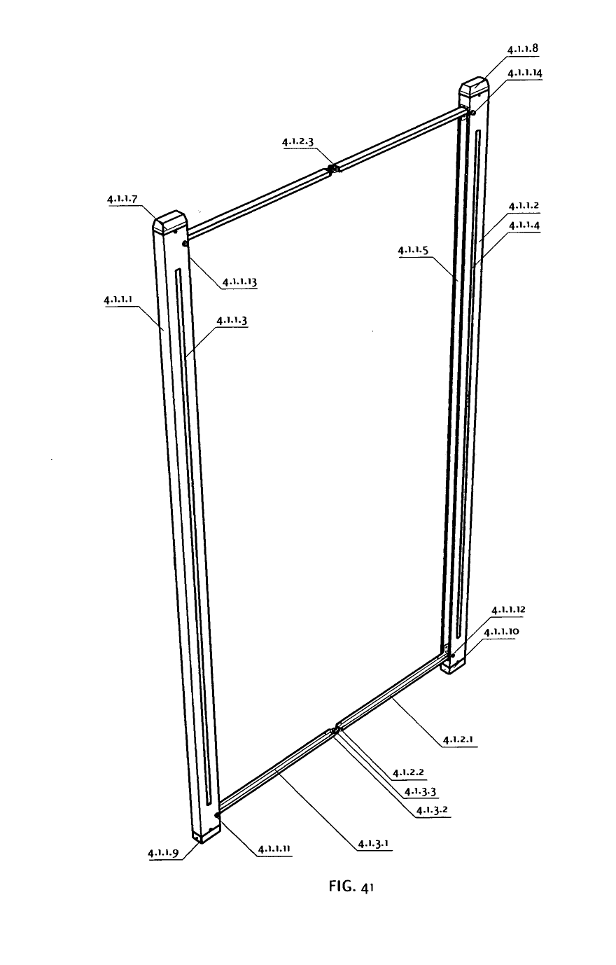

FIG. 41. In such Figure a view in front perspective of the Frame assembly is observed (4.1), where; a tubular body of rectangular section (4.1.1.1) in its frontal face has a continuous rectilinear slit (4.1.1.3); on this same face, in its upper end a cylindrical body is observed (4.1.1.13) and in its other end an analogous body (4.1.1.11). In the inner lateral face of the body (4.1.1.2) we can observe a greater slit (4.1.1.5); in its extreme lower portion, from its interior a laminar body (4.1.2.1) emerges opened in its upper part solidly joined to a laminar body (4.1.2.2) convergent in a flat body where a connector lodges (4.1.3.3); from here a symmetrical body (4.1.3.2) to (4.1.2.2) and contiguous to this one a body (4.1.3.1) symmetrical to (4.1.2.1). In the extreme upper face of (4.1.1.1) is a prismatic truncated body (4.1.1.7), and in its other end a rectangular body (4.1.1.9).

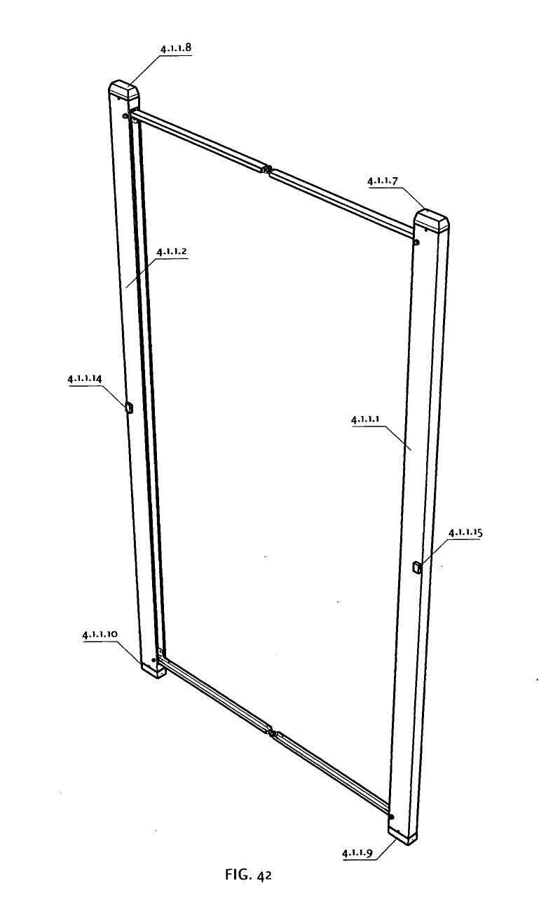

FIG. 42. In such Figure a view in rear perspective of the Frame assembly is observed (4.1), where; in the frontal face, is a polyhedral body (4.1.1.15) that emerges from the interior of this one; analog and symmetrical to this one, the polyhedral (4.1.1.14).

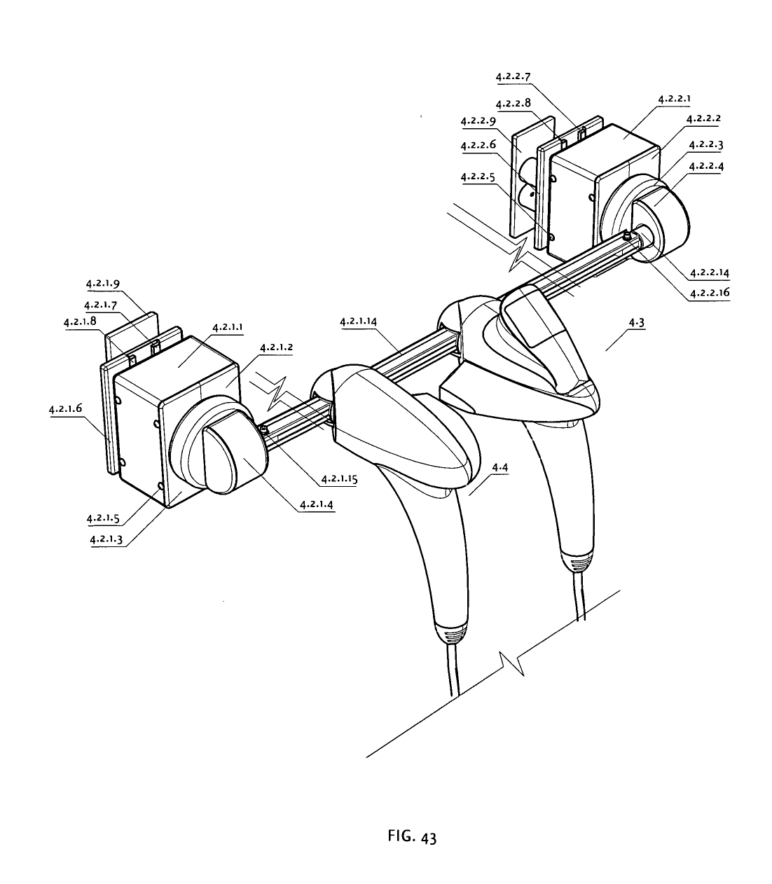

FIG. 43. In such Figure a view in front perspective of the Horizontal Axis assembly is observed (4.2), where; a drained polyhedral body (4.2.1.1), in whose frontal face a body of lower thickness is projected with its same section (4.2.1.2), and also from its frontal face, a cylindrical body is projected (4.2.1.3) of required diameter, and that, in turn, from its frontal face, a semi cylinder is projected (4.2.1.4). From this latter, and from its inner lateral face a tubular body of polygonal section is projected (4.2.1.14) by means of a cylindrical connector (4.2.1.15) that crosses it. At the end of (4.2.1.14) a cylindrical smaller body (4.2.2.14) is connected by means of a connector (4.2.1.16) also cylindrical, that crosses it; such body (4.2.2.14) is introduced in the semi cylindrical body (4.2.2.4), which in its flat base is the cylindrical body (4.2.2.3) to meet a polyhedral body of lower thickness (4.2.2.2) in whose rear part a drained body is projected with its same section (4.2.2.1). Behind this, we can notice at certain distance, a laminar body (4.2.2.5) which contains two rectangular magnetic bodies (4.2.2.7) and (4.2.2.8), of significantly smaller dimensions.

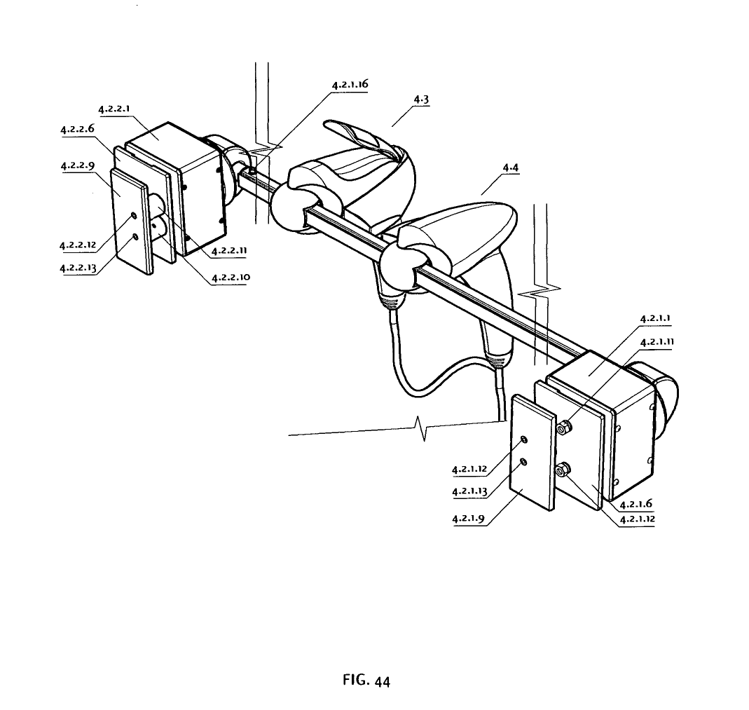

FIG. 44. In such Figure a view in rear perspective of the Horizontal Axis assembly is observed (4.2), where; equidistant to the body (4.2.2.6) a laminar body is projected (4.2.2.9) in whose space a cylindrical tubular body (4.2.2.11) replaces it and analogously (4.2.2.10).

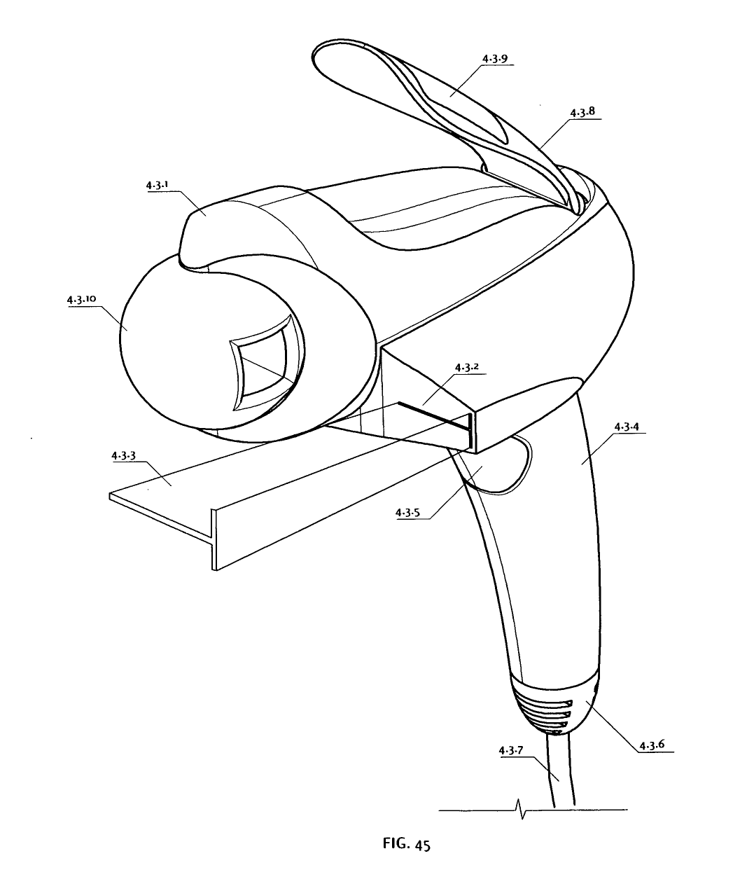

FIG. 45. In such Figure a view in front perspective of the Cartesian Master Manipulator assembly (4.3) is observed, where; a ellipsoid drained greater body (4.3.1), in its frontal part has a concavity followed by an angular cut with respect to the longitudinal plane, from which, in addition, a smaller body is laterally projected in whose frontal face a body (4.3.2) similar to a turned “T” is embedded, from which a luminous beam emerges (4.3.3) of equal section. Under the body (4.3.1), united, a decreasing body (4.3.4) of curved axis is projected, radious center that is towards the front of the assembly; in its end a body continues (4.3.6) that gives rounded ending; from whose lower portion a cylindrical flexo body extends (4.3.7). In the convexity of the body (4.3.1) a spherical body lodges (4.3.10) that is removed, cross-sectionally to the assembly, by a body of polygonal section. In its rear upper part a winding slit is observed, where an equally winding body extends (4.3.9), in which a smaller polyhedral body is embedded (4.3.8). On the body (4.3.4.) a winding body is embedded in its upper frontal portion (4.3.5).

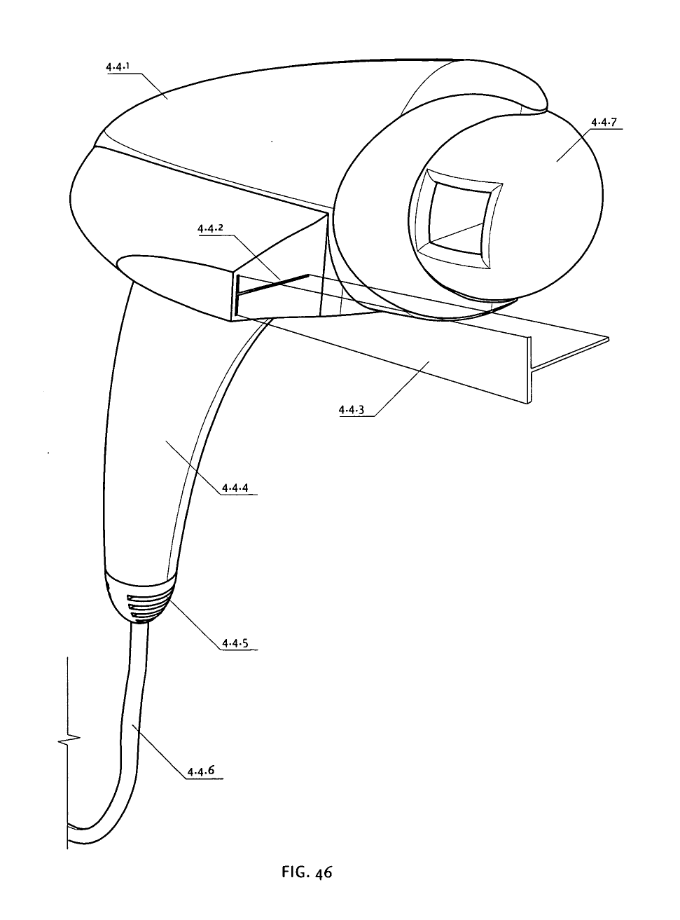

FIG. 46. In such Figure a view in front perspective of the Slave Cartesian Manipulator assembly is observed (4.4), where; a body (4.4.1) symmetrical to (4.3.1) and from whose frontal surface is a body (4.4.2), also symmetrical to (4.3.2), from where a luminous beam emerges (4.4.3).

The experts in the subject will understand that the foregoing refers solely to a preferred performance of the invention, which is susceptible of modifications, which does not imply to depart from the scope of the invention, defined by the claims that follow.

1-30. (canceled)

31. Autonomous digital transportable system for the performance of anthropometric measurements, which makes it easier to localize and collect the anthropometric points that are extracted from the person to be measured; storage process, digitalized and automatized processing and post-processing, which in at its phase during the measurement (FIG. 15.I) characterized because such system is formed by, a postural Rectifier 3) that receives the person to be measured (8), a Cartesian Frame (4) that is manipulated by the measurer (9),a computer application program (5), notebook or similar (5.1), wired or wireless data transmitters (6 and 7); mentioned Cartesian Frame (4), specifically the Cartesian manipulators (4.4 and 43) emit a bundle of light rays (433) that projects itself towards the measurable person (8), destined to refer to the localization of the required anthropometric point in a orthogonal and oblique way, this information processed by the sensors housed in the mentioned Cartesian manipulators (4.4 and 4.3) are pre-visualized in real time by means of a pivoting display (4.3.9), these anthropometric dimensions are digitally stored in the processor housed in vertical drained greater body (4.2.1.1), transmitted through wired or wireless data transmitters (6) to the computer application program (5) installed in a notebook or similar (5.1), performed during or subsequently to the measurement, avoiding besides improving the data visualization measurement's operating time.

32. System according to claim 1, characterized because the postural Rectifier (3) is composed of an upper support (3.1), a lower support (3.2) and a column (3.3), the latter, which joins and articulates them, being in turn, formed by Armrests (3.4) and Seat (3.5); this assembly allows to receive the postures of the person to be measured (8) and isolate him/her from the physical place where the measurement is intended to be performed.

33. System according to claim 2, characterized because the postural Rectifier (3), besides when the measured user (8) finds himself/herself over lower Support (3.2) collects the person's weight in order to transmit it with wire data transmitters (7) to the computer application program (5) installed in notebook or similar (5.1); avoids the utilization of auxiliary elements delaying the measurement process.

34. System according to Claim, characterized because the Upper Support (3.1), is a limited hinged positional receptor-rotational support.

35. System according to claim 2, characterized because the Lower Support (3.2), is a container-support, limited four-positional divergent receptor-rotational.

36. Device according to claim 2, characterized because the Column (3.3), is a pivoting support of two hinged vertebras, which form by means of its plates and planes.

37. Device according to claim 2, characterized because the Arm Support (3.4), is a support for upper parts of both children and adults, adjustable, extensible-rotating multi-postural.

38. Device according to claim 2, characterized because the Seat (3.5), is a seat both for children and adults, extensible-rotating, adjustable, multi-postural where the person shall adopt a seating position and then the devices shall adjust themselves to this posture.

39. Device according to claim 1, characterized because the Cartesian Frame (4) is composed of a frame (4.1) and a horizontal axis (4.2). The first one structures the system and the second one moves from parallel in an axial direction over the previous;
this assembly has a direct relation with the measurer user (9) avoiding the instrumental instability, making the measurement more precise and reliable; besides being capable of making it easier to localize 2 points in a sequential way and eventually simultaneously recording it.

40. Device according to claim 4, characterized because the assembly (4.2) can be easily slid in a greater axial direction in respect to (4) by means of the gesture intervention of the measuring user (9); by way of a Cartesian plane in a “Y” it is possible to secure the stability, reliability and therefore precision and decrease the measurement's frequency of error.

41. Device according to claim 4, characterized because the Cartesian Manipulators (4.2.3 and 4.2.4) may be independently and easily slid in a greater axial direction in respect to (4.2.1.14) by means of the gesture intervention of the measuring user (9); by way of a Cartesian plane in a “X” direction it is possible to secure the stability, reliability and therefore precision and decrease the measurement's frequency of error.

42. Device according to claim 4, characterized because the Frame (4.1), is a support container, drop-down, divergent, parallel support.

43. Device according to claim 4, characterized because the Horizontal Axis (4.2) is a container, movable in X and rotational Support (4.2.1.3).

44. Device according to claim 4, characterized because the Cartesian Master Manipulator (4.3), is a movable, rotational container that projects a visible bundle (4.3.3); has a body (4.3.8) that is pivoting to the major body, which shows the dimensions, major body in which the sensors that perform the measurement are housed, composed by a cohering body (4.3.4) in which there is a pulsing body (4.3.5); this assembly makes it easier to localize the first anthropometric point and visualize the latter's measures that shall be recorded, the weight, the vibration, angles and the one that is described next.

45. Device characterized because the Slave Cartesian Manipulator (4.4), is a movable, rotational container that projects a visible bundle (4.4.3); the latter allows to position the second anthropometric point, facilitated by the visible projected bundle (4.3.3).

46. Device according to claim 1, characterized because the Cartesian Frame (4) rotates in connection with the postural Rectifier (3); this allows the stable measuring of the user to be measured (8) in its 4 main projection views; frontal view, right lateral view, rear view, and left lateral view; avoiding the need to move the person in one of the desired postures.

47. Device according to claim 1, characterized because the postural Rectifier (3), may receive the posture of anthropometric attention or standing up posture of the person (8), by means of transparent laminar supports (3.3), the storing stage of the armrest (3.4) and the coplanarity of the stage of guarded of the scat (3.5); securing the required postural protocol.

48. Device according to claim 1, characterized because the postural Rectifier (3), may receive the posture of anthropometric modified or sitting posture of the person (8.1), by means of transparent laminar supports (3.3); the adjustability and possibility of multi postures offered by the armrest (3.4); and also, the adjustability and possibility of multi postures offered by the seat (3.5); securing the required postural protocol.

49. Device according to claim 1, characterized because the postural Rectifier (3) may receive the person's arm span posture (8.1), by means of transparent laminar supports (3.3); the adjustability and possibility of multi postures offered by the armrest (3.4) and the coplanarity of the storing stage of the seat (3.5); securing the required postural protocol.

50. Device in its phase before performing the measuring (FIG. 2 and FIG. 3) formed by the postural Rectifier (3) and the Cartesian Frame (4) that have respective protectors; for (3) the protector (1), and for (4) the protector (2), characterized because such postural Rectifier (3) and Frame (4) are individually transported in their before mentioned protectors that allows them to be moved in vertical heights and moved on ground level by people, and transported by vehicles of medium capacity, avoiding that its constitutive and susceptible of being damaged components are protected from impacts; then, it allows the set of instruments as two complementary units, to be taken to site and measure.
